(19) United States
(12) Patent Application Publication
(10) Pub. No.: US 2010/0159469 A1
(43) Pub. Date: Jun. 24, 2010

(54) Compositions and Methods for Breast Cancer Prognosis

(75) Inventors: Cole Harris, Albuquerque, NM (US); Lisa Davis, Albuquerque, NM (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: EXAGEN DIAGNOSTICS, INC., Albuquerque, NM (US)

(21) Appl. No.: 12/711,927

(22) Filed: Feb. 24, 2010

Related U.S. Application Data
(63) Continuation of application No. 11/112,908, filed on Apr. 22, 2005.
(60) Provisional application No. 60/564,758, filed on Apr. 23, 2004.
(60) Provisional application No. 60/575,978, filed on Jun. 1, 2004.
(60) Provisional application No. 60/631,702, filed on Nov. 30, 2004.
(60) Provisional application No. 60/633,826, filed on Dec. 7, 2004.

(51) Int. Cl. C12Q 1/68

(52) U.S. Cl. 435/6

(57) ABSTRACT

The present invention provides novel compositions and their use in classifying breast tumors.

### CROSS REFERENCE

The application is a continuation of U.S. Utility patent application Ser. No. 11/112,908 filed Apr. 22, 2005 which claims priority to U.S. Provisional Patent Application Ser. Nos. 60/564,758 filed Apr. 23, 2004; 60/575,978 filed Jun. 1, 2004; 60/631,702 filed Nov. 30, 2004; and 60/633,826 filed Dec. 7, 2004.

### FIELD OF THE INVENTION

The invention relates generally to the fields of nucleic acids, nucleic acid detection, cancer, and breast cancer.

### BACKGROUND

Breast cancer is the most common cancer in women and the second most common cause of cancer death in the United States. While germ line mutations in BRCA1 or BRCA2 genes predispose women with the mutations to breast cancer, only about 5-10% of breast cancers are associated with these breast cancer susceptibility genes. Currently employed clinical indicators of breast cancer prognosis are not accurate in identifying patients likely to have a favorable outcome. As a result, many more patients are subjected to adjuvant chemotherapy than will benefit from such treatment (US 20040058340 published Mar. 25, 2004).

Tumors not currently known to be associated with a germline mutation (“sporadic tumors”) constitute the majority of breast cancers (US 20040058340). Due to the increased morbidity and mortality if breast cancer is not detected early in its progression, considerable effort has been devoted to early detection of breast tumor development.

Breast cancer diagnosis typically requires histopathological proof of tumor presence. Histopathological examinations also provide information about prognosis and help guide selection of treatment regimens. Prognosis may also be established based upon parameters such as tumor size, tumor grade, the age of the patient, and lymph node metastasis (US 20040058340).

Accurate prognosis or determination of distant metastasis-free survival in breast cancer patients would permit selective administration of adjuvant chemotherapy, with women having poorer prognoses being given the most aggressive treatment.

The maturation of microarray technology has enabled the routine collection of genome-wide gene expression (RNA) data. Several authors have shown that microarray data collected from tumors may be useful in differential diagnosis, tumor staging and prognosis. The data produced by these studies ideally represents a valuable resource for the development of new diagnostics. However, current microarray technologies require sample collection and preparation steps that inhibit routine clinical adoption.

In contrast, DNA-based markers are commonly used in cancer diagnostics. Diagnostic implementations utilizing fluorescence in situ hybridization (FISH) and RT-PCR technology are in widespread use. New diagnostic products based on such accepted technology will more quickly find clinical acceptance.

It is established that specific genetic aberrations are often associated with clinical characteristics. Examples include the association of 1p/19q deletions in breast cancers with improved response to chemotherapy, and the association of 8q gain with poor prognosis in prostate cancer. Such aberrations have been detected with comparative genomic hybridization (“CGH”). However, the relationship between tumor karyotype and phenotype is often subtle, and may be difficult to determine from the typically available datasets consisting of low-resolution CGH data collected from a small number of samples.

Several studies have demonstrated the association of genetic aberrations with gene expression changes. In independent studies, Hyman et al (Cancer Res. 2002, Nov. 1:62(21):6240-5, 2002) and Pollack et al (Proc Natl Acad Sci USA 2002 Oct. 1; 99(20):12963-8) have found a strong relationship between high amplification and high expression in breast tumors. Crawley et al (Genome Biol. 2002; 3(12):RESEARCH0075. Epub 2002 Nov. 25) have reported on a data analysis method that accurately predicts regions of copy number aberrations in hepatocellular carcinomas using only gene expression data. These investigations support the notion that gene expression data can be used as a window to the underlying genetic defects, and thus the idea that a combined analysis of gene expression data and CGH copy number data with the aim of identifying DNA markers is viable.

Currently employed clinical indicators of breast cancer prognosis are not accurate in identifying patients likely to have a favorable outcome. As a result, many more patients are subjected to adjuvant chemotherapy than will benefit from such treatment. Thus, there remains a need in the art for better and more specific clinical predictors of breast cancer prognosis.

### SUMMARY OF THE INVENTION

The present invention provides novel compositions and their use in classifying breast tumors.

In a first aspect, the present invention provides compositions comprising or consisting of a breast cancer biomarker, wherein the breast cancer biomarker comprises or consists of between 2 and 35 different probe sets, wherein at least 40% of the different probe sets comprise or consist of one or more isolated polynucleotides that selectively hybridize to a genomic region selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the different probe sets in total selectively hybridize to at least two of the recited genomic regions.

In a second aspect, the invention provides compositions comprising a breast cancer biomarker comprising or consisting of between 2 and 42 different probe sets, wherein at least 40% of the different probe sets comprise or consist of one or more isolated polynucleotides that selectively hybridize to an isolated nucleic acid according to formula 1:

X1-X2-X3;

wherein X2 is a human genomic insert contained within a bacterial artificial chromosome (“BAC”), and is selected from the group consisting of SEQ ID NOS: 18-65 (see Table 1) or their complement, wherein X1 and X3 are independently 0-500 kB of human genomic nucleic acid flanking X2 in the human genome; and

wherein the different polynucleotide probe sets in total selectively hybridize to at least two non-overlapping nucleic acids according to formula 1.

In a third aspect, the present invention provides compositions comprising a breast cancer biomarker comprising or consisting of between 2 and 42 different probe sets, wherein at least 40% of the different probe sets comprise or consist of one or more isolated polynucleotides that selectively hybridize to a nucleic acid according to one of SEQ ID NO:1-17 or complements thereof; wherein the different probe sets in total selectively hybridize to at least two of the recited nucleic acids according to SEQ ID NO:1-17 or complements thereof.

In a further aspect, the present invention provides methods for classifying a breast tumor, comprising

(a) contacting a nucleic acid sample obtained from a subject having a breast tumor with polynucleotide probes that, in total, selectively hybridize to two or more genomic regions selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the contacting occurs under conditions to promote selective hybridization of the polynucleotides of the probe set to the two or more genomic regions;

(b) detecting formation of hybridization complexes;

(c) determining whether one or more of the genomic regions are present in an altered copy number in the nucleic acid sample; and

(d) correlating an altered copy number of one or more of the genomic regions with a breast cancer classification.

In a further aspect, the present invention provides methods for classifying a breast tumor comprising:

(a) contacting a mRNA-derived nucleic acid sample obtained from a subject having a breast tumor with nucleic acid probes that, in total, selectively hybridize to two or more nucleic acid targets selected from the group consisting of SEQ ID NO:1-17 or complements thereof; wherein the contacting occurs under conditions to promote selective hybridization of the nucleic acid probes to the nucleic acid targets, or complements thereof, present in the nucleic acid sample;

(b) detecting formation of hybridization complexes between the nucleic acid probes to the nucleic acid targets, or complements thereof, wherein a number of such hybridization complexes provides a measure of gene expression of the one or more nucleic acids according to SEQ ID NO:1-17; and

(c) correlating an alteration in gene expression of the one or more nucleic acids according to SEQ ID NO:1-17 relative to control with a breast cancer classification.

### DETAILED DESCRIPTION OF THE INVENTION

All publications, GenBank Accession references, references to bacterial artificial chromosome (“BAC”) accession numbers (sequences), patents and patent applications cited herein are hereby expressly incorporated by reference for all purposes.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

The present invention provides novel compositions and methods for their use in classifying breast tumors. As used herein, the term “classifying” means to determine one or more features of the breast tumor or the prognosis of a patient from whom a breast tissue sample is taken, including the following:
(a) Diagnosis of breast cancer (benign vs. malignant tumor);
(b) Metastatic potential, potential to metastasize to specific organs, or course of the tumor;
(c) Stage of the tumor;
(d) Patient prognosis in the absence of chemotherapy or hormonal therapy;
(e) Prognosis of patient response to treatment (chemotherapy, radiation therapy, and/or surgery to excise tumor)
(f) Predicted optimal course of treatment for the patient;
(g) Prognosis for patient relapse after treatment; and
(h) Patient life expectancy.

Prior art methods for marker-based prognosis have either focused on (a) analysis of expression or copy number of single genes or genomic regions, which is likely to be relevant for only a small subset of tumors; or on (b) analysis of a large array of many genes or genomic regions, which is impractical for use in clinical diagnostic laboratories and most research facilities.

The compositions of the present invention are identified herein as being useful markers for breast cancer classification, and are defined relative to the following nucleic acid sequences:

```
  1. GENBANK ACCESSION AL080059 (SEQ ID NO: 1)
  2. GENBANK ACCESSION NM_006281 (SEQ ID NO: 2):
Serine/threonine kinase 3 (“stk3”)
  3. GENBANK ACCESSION NM_000127 (SEQ ID NO: 3):
Exostoses 1 (“ext1”)
  4. GENBANK ACCESSION NM_006265 (SEQ ID NO: 4):
“rad21” (AKA “HR21”; “SCC1”; “NXP1”)
  5. GENBANK ACCESSION NM_001157 (SEQ ID NO: 5):
Annexin All (“anxa11”)
  6. GENBANK ACCESSION NM_000135 (SEQ ID NO:6):
Fanconi anemia complementation group A (“fanca”)
  7. GENBANK ACCESSION NM_007144 (SEQ ID NO: 7):
zinc finger protein 110 (RF110) (“znfl44”)
  8. GENBANK ACCESSION NM_003079 (SEQ ID NO: 8):
SWI/SNF related, matrix-associated, actin depen-
dent regulator of chromatin, subfamily e,
member 1 (“smarce”)
  9. GENBANK ACCESSION NM_001168 (SEQ ID NO: 9):
Baculoviral IAP-repeat containing 5 (“birc 5”;
AKA: “survivin”)
10. GENBANK ACCESSION NM_013374 (SEQ ID NO: 10):
Programmed cell death interacting protein
(PDCD6IP)
11. GENBANK ACCESSION NM_005310 (SEQ ID NO: 11):
Growth factor receptor-bound protein 7 (“GRB7”))
12. GENBANK ACCESSION NM_006804 (SEQ ID NO: 12):
Start domain containing 3 (“MLN64”)(also called
STARD3)
13. GENBANK ACCESSION NM_005536 (SEQ ID NO: 13):
inositol(myo)-1(or 4)-monophosphatase 1 (“IMPA1”)
14. GENBANK ACCESSION NM_002151 (SEQ ID NO: 14):
(“Hepsin”)
15. GENBANK ACCESSION X72631 (SEQ ID NO: 15):
hrev gene (“nr1d1”)
16. GENBANK ACCESSION NM_003457 (SEQ ID NO: 16):
zinc finger protein 207 (“ZNF207”)
17. GENBANK ACCESSION NM_000782 (SEQ ID NO: 17):
cytochrome P450, family 24, subfamily A,
polypeptide1 (“CYP24”)
```

While statistically significant, the inventors believe that the clinical diagnostic and prognostic utility of subsets of these seventeen gene markers is greater than the clinical diagnostic and prognostic utility of individual genes. Such combinations may better classify the complexity of genomic aberrations associated with particular phenotypes in breast cancer.

Many studies have demonstrated that when genomic regions are amplified (as in a tumor), the amplified region (“amplification”) most commonly consists of a number of genes, in spite of the tendency to describe an amplification in terms of a single gene. (Barlund et al., Cancer Res. 2000 Oct. 1; 60(19):5340-4; Kauraniemi et al., Cancer Res. 2001 Nov. 15; 61(22):8235-40; Pollack et al., 2002; Hyman et al., Cancer Res. 2002 Nov. 1; 62(21):6240-5; Monni et al., Proc. Natl. Acad. Sci. USA 2001 May 8; 98(10):5711-6). For example, a “her-2” amplification generally contains the her-2 gene and many flanking genes.

Physical distances between the genes used in these studies, as described in publicly available databases (for example, UCSC human genome www.genome.ucsc.edu) reveals that, while the sizes of amplifications vary among tumors, the size of an “average” amplification is reasonably estimated as at least 1 megabase.

Thus, in a first aspect, the present invention provides compositions comprising or consisting of a breast cancer biomarker, wherein the breast cancer biomarker comprises or consists of between 2 and 35 different probe sets, wherein at least 40% of the different probe sets comprise or consist of one or more isolated polynucleotides that selectively hybridize to a genomic region selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the different probe sets in total selectively hybridize to at least two of the recited genomic regions.

The recited genomic regions correspond to the chromosome band of the markers. The compositions of the invention can be used, for example, to provide improved breast cancer classification over that possible using prior art diagnostic and predictive compositions and methods. Table 1 provides a detailed summary of the individual markers, their GenBank accession number, genomic region at which the markers are located, and the names and SEQ ID NOS. of bacterial artificial chromosomes (“BAC”) that contain the marker (discussed in more detail below).

TABLE 1

| Accession | gene name and | | | BAC SEQ ID | Insert size |
|---|---|---|---|---|---|
| Number | SEQ ID NO | BAC name | Chromosome | NO | (nucleotides) |
| AL080059 | AL080059 | RP11-499K24 | 8q22.1 | (SEQ ID | 165883 |
| | (SEQ ID NO: 1) | | | NO: 18) | |
| NM_006281 | STK3 | RP11-159A16 | 8q22.2 | (SEQ ID | 212805 |
| | (SEQ ID NO: 2) | | | NO: 19) | |
| | | RP11-765K18 | 8q22.2 | (SEQ ID | 191331 |
| | | | | NO: 20) | |
| | | RP11-563E23 | 8q22.2 | (SEQ ID | 207908 |
| | | | | NO: 21) | |
| | | RP11-613I24 | 8q22.2 | (SEQ ID | 172147 |
| | | | | NO: 22) | |
| | | RP11-44N12 | 8q22.2 | (SEQ ID | 188682 |
| | | | | NO: 23) | |
| NM_000127 | EXT1 | RP11-486B24 | 8q24.11 | (SEQ ID | 150314 |
| | (SEQ ID NO: 3) | | | NO: 24) | |
| | | RP11-96F23 | 8q24.11 | (SEQ ID | 172781 |
| | | | | NO: 25) | |
| | | RP11-24C23 | 8q24.11 | (SEQ ID | 150173 |
| | | | | NO: 26) | |
| | | RP11-601K03 | 8q24.11 | (SEQ ID | 171247 |
| | | | | NO: 27) | |
| | | RP11-742D20 | 8q24.11 | (SEQ ID | 166020 |
| | | | | NO: 28) | |
| | | RP11-453O16 | 8q24.11 | (SEQ ID | 131855 |
| | | | | NO: 29) | |
| | | RP11-208A09 | 8q24.11 | (SEQ ID | 143389 |
| | | | | NO: 30) | |
| | | RP11-346L16 | 8q24.11 | (SEQ ID | 207600 |
| | | | | NO: 31) | |
| NM_006265 | RAD21 | RP11-367C15 | 8q24.11 | (SEQ ID | 193363 |
| | (SEQ ID NO: 4) | | | NO: 32) | |
| NM_001157 | ANXA11 | RP11-529C24 | 10q22.3 | (SEQ ID | 217623 |
| | (SEQ ID NO: 5) | | | NO: 33) | |
| | | RP11-668E21 | 10q22.3 | (SEQ ID | 197781 |
| | | | | NO: 34) | |
| NM_000135 | FANCA | RP11-354M24 | 16q24.3 | (SEQ ID | 127340 |
| | (SEQ ID NO: 6) | | | NO: 35) | |
| | | RP11-364G13 | 16q24.3 | (SEQ ID | 98345 |
| | | | | NO: 36) | |
| NM_007144 | ZNF144 | RP11-610D13 | 17q12 | (SEQ ID | 150481 |
| | (SEQ ID NO: 7) | | | NO: 37) | |
| | | RP11-607B02 | 17q12 | (SEQ ID | 171162 |
| | | | | NO: 38) | |
| | | RP11-15P20 | 17q12 | (SEQ ID | 179892 |
| | | | | NO: 39) | |
| NM_003079 | SMARCE1 | RP11-372J02 | 17q21.2 | (SEQ ID | 180862 |
| | (SEQ ID NO: 8) | | | NO: 40) | |
| NM_001168 | BIRC5 | RP11-141D15 | 17q25.3 | (SEQ ID | 177623 |
| | (SEQ ID NO: 9) | | | NO: 41) | |
| | | RP11-219G17 | 17q25.3 | (SEQ ID | 155515 |
| | | | | NO: 42) | |
| | | RP11-586J16 | 17q25.3 | (SEQ ID | 159660 |
| | | | | NO: 43) | |
| NM_013374 | PDCD6IP | RP11-683H06 | 3p23 | (SEQ ID | 150437 |
| | (SEQ ID NO: 10) | | | NO: 44) | |
| | | RP11-795G20 | 3p23 | (SEQ ID | 182314 |
| | | | | NO: 45) | |
| | | RP11-637B24 | 3p23 | (SEQ ID | 150491 |
| | | | | NO: 46) | |
| NM_005310 | GRB7 | RP11-563O04 | 17q12 | (SEQ ID | 166111 |
| | (SEQ ID NO: 11) | | | NO: 47) | |
| | | RP11-94L15 | 17q12 | (SEQ ID | 161726 |
| | | | | NO: 48) | |
| | | RP11-610O22 | 17q12 | (SEQ ID | 149419 |
| | | | | NO: 49) | |
| NM_006804 | STARD3 | RP11-689B15 | 17q12 | (SEQ ID | 170189 |
| | (SEQ ID NO: 12) | | | NO: 50) | |
| | | RP11-62N23 | 17q12 | (SEQ ID | 157224 |
| | | | | NO: 51) | |
| | | RP11-94L15 | 17q12 | (SEQ ID | 161726 |
| | | | | NO: 52) | |
| NM_005536 | IMPA1 | RP11-67F19 | 8q21.13 | (SEQ ID | 191343 |
| | (SEQ ID NO: 13) | | | NO: 53) | |
| | | RP11-34M16 | 8q21.13 | (SEQ ID | 150450 |
| | | | | NO: 54) | |
| NM_002151 | HEPSIN | RP11-737K14 | 19q13.12 | (SEQ ID | 193789 |
| | (SEQ ID NO: 14) | | | NO: 55) | |
| | | RP11-233I16 | 19q13.12 | (SEQ ID | 150468 |
| | | | | NO: 56) | |
| X72631 | NR1D1 | RP11-278E15 | 17q21.1 | (SEQ ID | 161994 |
| | (SEQ ID NO: 15) | | | NO: 57) | |
| | | RP11-735P18 | 17q21.1 | (SEQ ID | 170285 |
| | | | | NO: 58) | |
| | | RP11-749I16 | 17q21.1 | (SEQ ID | 168656 |
| | | | | NO: 59) | |
| NM_003457 | ZNF207 | RP11-299H03 | 17q11.2 | (SEQ ID | 171427 |
| | (SEQ ID NO: 16) | | | NO: 60) | |
| | | RP11-634A23 | 17q11.2 | (SEQ ID | 159497 |
| | | | | NO: 61) | |
| NM_000782 | CYP24A1 | RP11-92B18 | 20q13.2 | (SEQ ID | 170508 |
| | (SEQ ID NO: 17) | | | NO: 62) | |
| | | RP11-310L22 | 20q13.2 | (SEQ ID | 149111 |
| | | | | NO: 63) | |
| | | RP11-234K07 | 20q13.2 | (SEQ ID | 157230 |
| | | | | NO: 64) | |
| | | RP11-114N21 | 20q13.2 | (SEQ ID | 173115 |
| | | | | NO: 65) | |

Thus, the compositions of each aspect and embodiment of the present invention are useful, for example, in classifying human breast cancers. The compositions can be used, for example, to identify one or more genomic regions as present in an abnormal copy number (for example, more than two copies of the gene per cell in a chromosome spread or fewer than two copies) in a nucleic acid sample from a human specimen, such as breast tissue from a human subject, which provides a classification of the breast tumor as discussed above and below. Alternatively, certain embodiments of the compositions (as discussed in more detail below) can be used to determine the expression levels in tissue of the mRNA encoded by the genes recited above.

The compositions according to each of the aspects and embodiments of the invention provide an improvement over prior art breast cancer classification compositions, which require a much larger number of probes to classify a breast tumor, and do so with reduced accuracy compared to the breast cancer biomarker of the present invention. As a result, the compositions of the present invention are much more amenable to use in clinical diagnostic and prognostic testing than are prior art compositions and their use in methods for breast cancer classification.

The term “polynucleotide” as used herein with respect to each aspect and embodiment of the invention refers to DNA or RNA, preferably DNA, in either single- or double-stranded form. It includes the recited sequences as well as their complementary sequences, which will be clearly understood by those of skill in the art. The term “polynucleotide” encompasses nucleic acids containing known analogues of natural nucleotides which have similar or improved binding properties, for the purposes desired, as the disclosed polynucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup (1997) Biochemistry 36:8692-8698), and benzylphosphonate linkages, as discussed in U.S. Pat. No. 6,664,057; see also Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press).

An “isolated” polynucleotide as used herein for all of the aspects and embodiments of the invention is one which is free of sequences which naturally flank the polynucleotide in the genomic DNA of the organism from which the nucleic acid is derived, except as specifically described herein. Preferably, an “isolated” polynucleotide is substantially free of other cellular material, gel materials, vector linker sequences, and culture medium when produced by recombinant techniques, or substantially free of chemical precursors or other chemicals when chemically synthesized. The polynucleotides of the invention may be isolated from a variety of sources, such as by PCR amplification from genomic DNA, mRNA, or cDNA libraries derived from mRNA, using standard techniques; or they may be synthesized in vitro, by methods well known to those of skill in the art, as discussed in U.S. Pat. No. 6,664,057 and references disclosed therein. Synthetic polynucleotides can be prepared by a variety of solution or solid phase methods. Detailed descriptions of the procedures for solid phase synthesis of polynucleotide by phosphite-triester, phosphotriester, and H-phosphonate chemistries are widely available. (See, for example, U.S. Pat. No. 6,664,057 and references disclosed therein). Methods to purify polynucleotides include native acrylamide gel electrophoresis, and anion-exchange HPLC, as described in Pearson (1983) J. Chrom. 255:137-149. The sequence of the synthetic polynucleotides can be verified using standard methods.

As used herein with respect to all aspects and embodiments of the invention, a “probe set” refers to a group of one or more polynucleotides that each selectively hybridize to the same target (for example, a specific genomic region or mRNA) that can be used, for example, in breast cancer classification. Thus, a single “probe set” may comprise any number of different isolated polynucleotides that selectively hybridize to a given target. For example, a probe set that selectively hybridizes to SEQ ID NO:10 may comprise one or more probes for a single 100 nucleotide segment of SEQ ID NO:10 and also a different 100 nucleotide segment of SEQ ID NO:10, or both these in addition to a separate 10 nucleotide segment of SEQ ID NO:10, or 500 different 10 nucleotide segments of SEQ ID NO:10 (such as, for example, fragmenting a larger probe into many individual short polynucleotides). Those of skill in the art will understand that many such permutations are possible.

In this first aspect, the breast cancer biomarker can be any breast cancer biomarker that comprises or consists of between 2 and 35 probe sets as defined herein, wherein at least 40% of the probe sets comprise or consist of one or more isolated polynucleotides that selectively hybridize to one of the recited genomic regions. Such breast cancer biomarkers thus can contain other probe sets for use in breast cancer classification, diagnosis, or analysis, so long as at least 40% of the probe sets comprise one or more isolated polynucleotides that selectively hybridize to one of the recited genomic regions, and so long as no more than 35 probe sets are present in the breast cancer biomarker.

In preferred embodiments of the first aspect of the invention, at least 45%, 50%, 55%, 60%, 65%, 70%, 80%, 85%, 90%, 95%, or 100% of the probe sets comprise or consist of one or more isolated polynucleotides that selectively hybridize to one of the recited genomic regions. As will be apparent to those of skill in the art, as the percentage of probe sets that comprise or consist of one or more isolated polynucleotides that selectively hybridize to one of the recited genomic regions increases, the maximum number of probe sets in the breast cancer biomarker will decrease accordingly. Thus, for example, where at least 80% of the probe sets comprise or consist of one or more isolated polynucleotides that selectively hybridize to one of the recited genomic regions, the breast cancer biomarker will consist of between 2 and 17 probe sets. Those of skill in the art will recognize the various other permutations encompassed by the compositions according to the various aspects of the invention.

In a further preferred embodiment of the first aspect of the invention, the breast cancer biomarker comprises or consists of between 2 and 35 different probe sets, wherein the different probe sets in total selectively hybridize at least the following genomic regions: 20q13.2 (includes CYP24) and 3p23 (includes PDCD6IP). This embodiment (“HR+ composition 1”) is demonstrated herein to be particularly effective for classifying hormone receptor positive breast tumors, where “hormone receptor positive” is defined throughout the application as positive for either or both of estrogen receptors and progesterone receptors. In this embodiment, it is further preferred that the isolated polynucleotides in total selectively hybridize to a region of 17q25.3 (includes BIRC5). In various further preferred embodiments of HR+ composition 1, at least 45%, 50%, 55%, 60%, 65%, 70%, 80%, 85%, 90%, 95%, or 100% of the probe sets comprise or consist of one or more isolated polynucleotides that selectively hybridize to one of these 2 or 3 recited genomic regions.

In a further preferred embodiment of the first aspect of the invention, the breast cancer biomarker comprises or consists of between 2 and 35 different probe sets, wherein the different probe sets in total selectively hybridize to at least the following genomic regions: 17q21.2 (includes SMARCE 1) and 17q21.1 (includes NR1D1). This embodiment (“HR− composition 1”) is demonstrated herein to be particularly effective for classifying hormone receptor negative breast tumors, where “hormone receptor negative” is defined throughout the application as negative for either or both of estrogen receptors and progesterone receptors. In this embodiment, it is further preferred that the polynucleotides in total selectively hybridize to the region of 17q25.3 (includes BIRC5). In these various HR− composition 1 embodiments, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of these 2 or 3 recited genomic regions.

The composition of each aspect and embodiment of the invention may further comprise other polynucleotide components that are beneficial for use in combination with the breast cancer biomarker, such as competitor nucleic acids and other control sequences (such as sequences to provide a standard of hybridization for comparison, etc.) Such other polynucleotide components are not probe sets for purposes of the compositions and methods of the invention. The compositions may optionally comprise other components, including but not limited to buffer solutions, hybridization solutions, detectable labels, and reagents for storing the nucleic acid compositions.

As used herein with respect to each aspect and embodiment of the invention, the term “selectively hybridizes” means that the isolated polynucleotides bind to target genomic region or other target to form a hybridization complex, and minimally or not at all to other sequences. The specific hybridization conditions used will depend on the length of the polynucleotide probes employed, their GC content, as well as various other factors as is well known to those of skill in the art. (See, for example, Tijssen (1993) Laboratory Techniques in Biochemistry and Molecular Biology—Hybridization with Nucleic Acid Probes part I, chapt 2, “Overview of principles of hybridization and the strategy of nucleic acid probe assays,” Elsevier, N.Y. (“Tijssen”)). In one embodiment, stringent hybridization and wash conditions are selected to be about 5° C. lower than the thermal melting point (Tm) for the specific polynucleotide at a defined ionic strength and pH. The Tm is the temperature (under defined ionic strength and pH) at which 50% of the target sequence hybridizes to a perfectly matched probe. High stringency conditions are selected to be equal to the Tm for a particular polynucleotide probe. An example of stringent conditions are those that permit selective hybridization of the isolated polynucleotides to the genomic or other target nucleic acid to form hybridization complexes in 0.2×SSC at 65° C. for a desired period of time, and wash conditions of 0.2×SSC at 65° C. for 15 minutes. It is understood that these conditions may be duplicated using a variety of buffers and temperatures. SSC (see, e.g., Sambrook, Fritsch, and Maniatis, in: Molecular Cloning, A Laboratory Manual, Cold Spring Harbor Laboratory Press, 1989) is well known to those of skill in the art, as are other suitable hybridization buffers.

In various preferred embodiments of this first aspect of the invention, the breast cancer biomarker includes three, four, five, six, seven, eight, nine, ten, eleven, twelve, thirteen, or fourteen different probe sets that comprise or consist of one or more isolated polynucleotides that selectively hybridize to a genomic region selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2, wherein the different probe sets in total selectively hybridize to at least three, four, five, six, seven, eight, nine, ten, eleven, twelve, thirteen, or fourteen of the recited genomic regions. In each of these embodiments, it is further preferred that at least 45%, 50%, 55%, 60%, 65%, 70%, 80%, 85%, 90%, 95%, or 100% of the probe sets for a given breast cancer biomarker comprise or consist of one or more isolated polynucleotides that selectively hybridize to one of the recited genomic regions.

In each of the aspects and embodiments of the compositions and methods of the present invention, it is further preferred that the isolated polynucleotides are labeled with a detectable label. In a preferred embodiment, the detectable labels on the isolated polynucleotides in one probe set are all the same, and are distinguishable from the detectable labels on the isolated polynucleotides in the other probe sets in a given breast cancer biomarker. Such labeling of the isolated polynucleotides facilitates differential determination of the signals from different probe sets in a given breast cancer biomarker. Useful detectable labels include but are not limited to radioactive labels such as ³²P, ³H, and ¹⁴C; fluorescent dyes such as fluorescein isothiocyanate (FITC), rhodamine, lanthanide phosphors, Texas red, and ALEXIS™ (Invitrogen), CY™ dyes (Amersham); (Spectrum Dyes, Abbott Labs), electron-dense reagents such as gold; enzymes such as horseradish peroxidase, beta-galactosidase, luciferase, and alkaline phosphatase; colorimetric labels such as colloidal gold; magnetic labels such as those sold under the mark DYNABEADS™; biotin; dioxigenin; or haptens and proteins for which antisera or monoclonal antibodies are available. The label can be directly incorporated into the polynucleotide, or it can be attached to a molecule which hybridizes or binds to the polynucleotide. The labels may be coupled to the isolated polynucleotides by any means known to those of skill in the art. In a various embodiments, the isolated polynucleotides are labeled using nick translation, PCR, or random primer extension (see, e.g., Sambrook et al. supra). Methods for detecting the label include, but are not limited to spectroscopic, photochemical, biochemical, immunochemical, physical and chemical techniques.

Those of skill in the art are aware that multiple resources are available to identify specific nucleotide sequences associated with the genomic regions discussed above. In one example, such sequences can be found as follows:

Go to the UCSC web site,
http://genome.ucsc.edu/index.html?org=Human. At this site, select the Genome Browser on the menu at the left. Then in the “position” field enter, (in this format, e.g. for chromosome 16p13): 16:11,000,000-12,000,000 and then select “jump” (position entries have to be either by gene name, clone name, accession number, etc. or base pair position, usually in millions) Once the image of the chromosome is in view, which has the base pairs at the top of the image, and the chromosome bands immediately below, the navigation tools can be used to zoom in or out, move to the left or right as necessary. To get to the sequence itself (for 16p13, as an example), select the band designation within the image, which leads to the “Chromosome Bands Localized by FISH Mapping Clones (p13.2)” page, which has the “View DNA for this feature” button. Choose the “View DNA . . . ” button which leads to the “Get DNA in Window”. At the bottom of that page choose the “Get DNA” button, and the sequence appears. At the very top of the sequence page the exact base pairs are shown.

Those of skill in the art will understand how to apply the present disclosure to identify the nucleotide sequences of other genomic regions of interest disclosed herein.

In a second aspect, the invention provides compositions comprising a breast cancer biomarker comprising or consisting of between 2 and 42 different probe sets, wherein at least 40% of the different probe sets comprise or consist of one or more isolated polynucleotides that selectively hybridize to an isolated nucleic acid according to formula 1:

X1-X2-X3;

wherein X2 is a human genomic insert contained within a bacterial artificial chromosome (“BAC”), and is selected from the group consisting of SEQ ID NOS: 18-65 (see Table 1) or their complement, wherein X1 and X3 are independently 0-500 kB of human genomic nucleic acid flanking X2 in the human genome; and

wherein the different polynucleotide probe sets in total selectively hybridize to at least two non-overlapping nucleic acids according to formula 1.

BAC sequence information is provided below in Table 2 (and as provided in Table 1), as well as the figures noted in Table 2.

TABLE 2

| | | |
|---|---|---|
| (a) | AL080059: | RP11-499K24 |
| (b) | STK3: | RP11-159A16 |
| | | RP11-765K18 |
| | | RP11-563E23 |
| | | RP11-613I24 |
| | | RP11-44N12 |
| (c) | EXT1: | RP11-486B24 |
| | | RP11-96F23 |
| | | RP11-24C23 |
| | | RP11-601K03 |
| | | RP11-742D20 |
| | | RP11-453O16 |
| | | RP11-208A09 |
| | | RP11-346L16 |
| (d) | RAD21: | RP11-367C15 |
| (e) | ANXA11: | RP11-529C24 |
| | | RP11-668E21 |
| (f) | MLN64: | RP11-689B15 |
| | (STARD3) | RP11-62N23 |
| | | RP11-94L15 |
| (g) | CYP24A1: | RP11-92B18 |
| | | RP11-310L22 |
| | | RP11-234K07 |
| | | RP11-114N21 |
| (h) | IMPA1: | RP11-67F19 |
| | | RP11-34M16 |
| (i) | GRB7: | RP11-563O04 |
| | | RP11-94L15 |
| | | RP11-610O22 |
| (j) | PDCD6IP: | RP11-683H06 |
| | | RP11-795G20 |
| | | RP11-637B24 |
| (k) | BIRC5: | RP11-141D15 |
| | | RP11-219G17 |
| | | RP13-586J16 |
| (l) | SMARCE1: | RP11-372J02 |
| (m) | ZNF144: | RP11-610D13 |
| | | RP11-607B02 |
| | | RP11-15P20 |
| (n) | FANCA: | RP11-354M24 |
| | | RP11-364G13 |
| (o) | Hepsin: | RP11-737K14 |
| | | RP11-233I16 |
| (p) | NR1D1: | RP11-278E15 |
| | | RP11-735P18 |
| | | RP11-749I16 |
| (q) | ZNF207: | RP11-299H03; and |
| | | RP11-634A23. |

The nucleic acids disclosed above in the “X2” group are the human genomic sequences encompassing the marker genes (and portions of the genomic regions of the first aspect of the invention) discussed above, cloned into BAC vectors. (See Table 1) As will be apparent to those of skill in the art in reviewing Table 1, genomic regions for each of the cloned markers for breast cancer classification described above (SEQ ID NO:1-17) are present in the BAC inserts listed within the “X2” groups above. For some of the 17 cloned markers, multiple overlapping BAC insert sequences are provided (see Tables 1 and 2).

According to this second aspect of the invention, the different polynucleotide probe sets in total selectively hybridize to at least two non-overlapping nucleic acids according to Formula 1 (ie: at least two of (a)-(q) in Table 2).

In various preferred embodiments of this second aspect of the invention, the breast cancer biomarker comprises or consists of three, four, five, six, seven, eight, nine, ten, eleven, twelve, thirteen, fourteen, fifteen, sixteen, or seventeen different probe sets that selectively hybridize to an isolated nucleic acid sequence according to formula 1, or its complement. In each of these embodiments, it is further preferred that at least 45%, 50%, 55%, 60%, 65%, 70%, 80%, 85%, 90%, 95%, or 100% of the probe sets for a given breast cancer biomarker comprise or consist of one or more isolated polynucleotides that selectively hybridize to a nucleic acid according to formula 1, or its complement, wherein the different polynucleotide probe sets in total selectively hybridize to at least three, four, five, six, seven, eight, nine, ten, eleven, twelve, thirteen, fourteen, fifteen, sixteen, or seventeen non-overlapping nucleic acids according to formula 1.

As will be apparent to those of skill in the art, as the percentage of probe sets that comprise or consist of one or more isolated polynucleotides that selectively hybridize to a nucleic acid according to formula 1, or its complement, the maximum number of probe sets in the breast cancer biomarker will decrease accordingly. Thus, for example, where at least 80% of the probe sets comprise or consist of one or more isolated polynucleotides that selectively hybridize to a nucleic acid according to formula 1, or its complement, the breast cancer marker will consist of between 2 and 21 probe sets. Those of skill in the art will recognize the various other permutations encompassed by the compositions according to the various embodiments of the second aspect of the invention.

In a further preferred embodiment of the second aspect of the invention, the different probe sets comprise or consist of one or more isolated polynucleotides that in total selectively hybridize to at least two different nucleic acids according to Formula I having X2 groups as follows:

a) one or more of SEQ ID NO:62-65 (includes CYP24), or complements thereof; and

b) one or more of SEQ ID NO:44-46 (includes PDCD6IP), or complements thereof.

This embodiment (“HR+ composition 2”) is demonstrated herein to be particularly effective for classifying hormone receptor positive breast tumors, where “hormone receptor positive” is defined as positive for either or both of estrogen receptors and progesterone receptors. In a further embodiment of the HR+ composition 2, the composition includes a probe set comprising or consisting of isolated polynucleotides that selectively hybridize to SEQ ID NO:41-43 (includes BIRC5), or complements thereof.

In various further preferred embodiments of HR+ composition 2, the different probe sets comprise or consist of one or more isolated polynucleotides that in total selectively hybridize to one or more nucleic acid from each of the following groups:

(a) SEQ ID NOS: 256-327 (unique sequence probes from the CYP24 containing BAC), or complements thereof; and

(b) SEQ ID NOS: 160-255 (unique sequence probes from the PDCD6IP containing BAC), or complements thereof.

Nucleic acids in group (a) are unique sequence regions from the BAC including CYP24, and nucleic acids in group (b) are unique sequence regions from the BAC including PDCD6IP. Thus, this embodiment of HR+ composition 2 provides unique sequence probes for use if the methods of the invention, which obviates the need for competitor DNA in hybridization assays. In a further embodiment, the unique sequence probes for HR+ composition includes a probe set comprising or consisting of isolated polynucleotides that selectively hybridize to one or more nucleic acid from the groups:

(c) SEQ ID NOS:416-511 (unique sequence probes from the BIRC5 containing BAC), or complements thereof.

In these various embodiments of HR+ composition 2, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of the 2 or 3 recited nucleic acids.

In a further preferred embodiment of the second aspect of the invention, the different probe sets comprise or consist of one or more isolated polynucleotides that in total selectively hybridize to at least two different nucleic acids according to Formula I having X2 groups as follows:

(a) one or more of SEQ ID NO:57-59 (includes NR1D1), or complements thereof; and

(b) SEQ ID NO:40 (includes SMARCE1), or complements thereof.

This embodiment (“HR− composition 2”) is demonstrated herein to be particularly effective for classifying hormone receptor negative breast tumors, where “hormone receptor negative” is defined as negative for either or both of estrogen receptors and progesterone receptors. In a further embodiment of the HR− composition 2, the composition includes a probe set comprising or consisting of isolated polynucleotides that selectively hybridize to one or more of SEQ ID NO:41-43 (includes BIRC5), or complements thereof.

In various further preferred embodiments of HR− composition 2, the different probe sets in total selectively hybridize to one or more nucleic acid from each of the following groups:

(a) SEQ ID NOS:328-415 (unique sequence probes from the NR1D1-containing BAC), or complements thereof; and

(b) SEQ ID NOS:66-159 (unique sequence probes from the SMARCE-containing BAC), or complements thereof.

Nucleic acids in group (a) are unique sequence regions from the BAC including (NR1D1, and nucleic acids in group (b) are unique sequence regions from the BAC including SMARCE 1. Thus, this embodiment of HR+ composition 2 provides unique sequence probes for use if the methods of the invention, which obviates the need for competitor DNA in hybridization assays. In a further embodiment, the unique sequence probes for HR− composition 2 the different probe sets in total selectively hybridize to one or more of: (c) SEQ ID NOS:416-511 (unique sequence probes from the BIRC5-containing BAC), or complements thereof.

In these embodiments of HR− composition 2, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of these 3 recited nucleic acids.

In a further preferred embodiment of each of the above embodiments of the second aspect of the invention, X1 and X3 are 0-400 kb; 0-300 kb; 0-200 kb. 0-100 kb; or 0 kb.

In a preferred embodiment of the various embodiments of the second aspect of the invention, the different probe sets of a breast cancer biomarker comprise or consist of one or more polynucleotides of at least 10 nucleotides of a nucleic acid according to formula 1, or complements thereof. In a further preferred embodiment, the different probe sets of a breast cancer biomarker comprise or consist of one or more polynucleotide of at least 10 nucleotides of a nucleic acid selected from the group consisting of SEQ ID NO:18 to SEQ ID NO: 511, or complements thereof.

In various further preferred embodiments of each of the embodiments of the first and second aspects of the invention, and related aspects and embodiments described below, the polynucleotides in the probe sets independently comprise or consist of at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10,000; 15,000; 20,000; 25,000; 30,000; 35,000; 40,0000; 45,000; 50,000; 60,000; 70,000; 80,000; 90,000; 100,000; 110,000; 120,000; 130,000; 140,000; 150,000; 160,000; 170,000; 180,000; 190,000; 200,000; 210,000; or 220,000 nucleotides of the relevant sequence, or complements thereof.

The BACS disclosed herein are as defined on the University of California at Santa Cruz (UCSC) Genome Browser on Human April 2003 Freeze and are available from the Children's Hospital Oakland Research Institute at www.bacpac.chori.org. The human genomic inserts cloned into the BACS disclosed herein range in size from approximately 150 kB to 220 in length.

As of March of 2004, detailed information on the BACS is available by going to the web site for the Children's Hospital Oakland Research Institute at www.bacpac.chori.org and clicking on the link to “Human ‘32K’ BAC Re-array” under the products menu. From this page, www.bcgsc.ca/lab/mapping/bacrearray/human/ provides a link to the Genome Sciences Centre web page. From this page, go to the Annotations box and find the further box for “Browse clone set”. Within that box is a link to the UCSC Genome Browser; click on the link that says “available,” which takes you to http://genome.ucsc.edu/cgi-bin/hgTracks, where detailed BAC information, such as that provided in the accompanying figures, can be found. The BACS can be found by searching by BAC name or by gene name. The sequence of the human genomic insert cloned in a BAC of interest can be found at http://genome.ucsc.edu/cgi-bin/hgTracks. Once the BAC of interest has been found in the database, as described above, the sequence of each BAC be found by “clicking” on the name of the BAC. The first click connects to a “Custom Track” for that BAC. On the Custom Track page there is an option called “View DNA for this feature”, which is a link to the “Get DNA” window, for that specific BAC. On the “Get DNA” page, the “Get DNA” button retrieves the complete DNA sequence for that BAC clone. Furthermore, sequences flanking the BAC of interest can also be retrieved from the “Get DNA” page by using “Sequence Retrieval Option”: the number of bases desired both upstream and downstream of the BAC are entered and, and those flanking sequences are then retrieved along with the sequence of the BAC itself. Furthermore, the detailed information on the BACS provided herein discloses the genomic location in terms of base pair position of the human genomic insert cloned in BACS as of the Human April 2003 Freeze.

As will be understood by those of skill in the art, the human genome sequence is frequently updated, with the updates made available to the public. Those of skill in the art will thus be able to identify the sequences flanking the human genomic insert cloned in a BAC of interest disclosed herein by accessing the human genome information (for example, at http://genome.ucsc.edu/). Therefore, the “flanking sequences” as recited herein refer to flanking sequences as disclosed on the web sites provided above, as well as updates thereto. For example, one can go to the UCSC Genome Browser site as disclosed above and review the BAC information as of the Human April 2003 Freeze to get the relative base pair position on the chromosome that the human genomic insert cloned in a BAC of interest was derived from. By reviewing the human genome sequence data available at as of the Human April 2003 Freeze (as described above), one of skill in the art can obtain the nucleic acid sequences flanking the human genomic insert cloned in a BAC of interest disclosed herein. Those of skill in the art can further use this sequence to identify sequences flanking the human genomic insert cloned in a BAC of interest from this same site as currently updated in the human genome sequence, or from other similar sites that provide human genome sequence information.

In a third aspect, the present invention provides compositions comprising a breast cancer biomarker comprising or consisting of between 2 and 42 different probe sets, wherein at least 40% of the different probe sets comprise or consist of one or more isolated polynucleotides that selectively hybridize to a nucleic acid according to one of SEQ ID NO:1-17 or complements thereof; wherein the different probe sets in total selectively hybridize to at least two of the recited nucleic acids according to SEQ ID NO:1-17 or complements thereof.

In various preferred embodiments of the third aspect of the invention, the composition comprises a breast cancer biomarker comprising or consisting of three, four, five, six, seven, eight, nine, ten, eleven, twelve, thirteen, fourteen, fifteen, sixteen, or seventeen different probe sets that comprise of consist of one or more isolated polynucleotides that selectively hybridize to a nucleic acid according to one of SEQ ID NO:1-17 or complements thereof, wherein different probe sets in total selectively hybridize to at least three, four, five, six, seven, eight, nine, ten, eleven, twelve, thirteen, fourteen, fifteen, sixteen, or seventeen of the recited nucleic acids according to SEQ ID NO:1-17 or complements thereof. In each of these embodiments, it is further preferred that at least 45%, 50%, 55%, 60%, 65%, 70%, 80%, 85%, 90%, 95%, or 100% of the probe sets for a given breast cancer biomarker comprise or consist of one or more isolated polynucleotides that selectively hybridize to a nucleic acid according to SEQ ID NO:1-17, or complements thereof. As will be apparent to those of skill in the art, as the percentage of probe sets that comprise or consist of one or more isolated polynucleotides that selectively hybridize to a nucleic acid according to SEQ ID NO:1-17, or complements thereof, the maximum number of probe sets in the breast cancer biomarker will decrease accordingly. Thus, for example, where at least 80% of the probe sets comprise or consist of one or more isolated polynucleotides that selectively hybridize to a nucleic acid according to SEQ ID NO:1-17, or complements thereof, the breast cancer biomarker will consist of between 2 and 21 probe sets. Those of skill in the art will recognize the various other permutations encompassed by the compositions according to the various embodiments of the third aspect of the invention.

In a preferred embodiment of the various embodiments of the third aspect of the invention, the different probe sets of a breast cancer biomarker comprise or consist of one or more polynucleotides of at least 10 nucleotides of a nucleic acid according to SEQ ID NO:1-17, or complements thereof.

In a further preferred embodiment of the third aspect of the invention, the different probe sets comprise or consist of isolated polynucleotides that in total selectively hybridize to at least SEQ ID NO:17 (CYP24), and SEQ ID NO:10 (PDCD6IP), or complements thereof. This embodiment (“HR+ composition 3”) is demonstrated herein to be particularly effective for classifying hormone receptor positive breast tumors, where “hormone receptor positive” is defined as positive for either or both of estrogen receptors and progesterone receptors. In this embodiments of HR+ composition 3, it is further preferred that the composition includes a probe set comprising or consisting of isolated polynucleotides that selectively hybridize to SEQ ID NO:9 (BIRC5), or complements thereof. In these various embodiments of HR+ composition 3, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of these 2 or 3 recited nucleic acids, or complements thereof.

In a further preferred embodiment of the third aspect of the invention, the different probe sets in total selectively hybridize to at least SEQ ID NO:15 (NR1D1 and SEQ ID NO:8 (SMARCE 1), or complements thereof. This embodiment (“HR− composition 3”) is demonstrated herein to be particularly effective for classifying hormone receptor negative breast tumors, where “hormone receptor negative” is defined as negative for either or both of estrogen receptors and progesterone receptors. In this embodiment of HR− composition 3, it is further preferred that the composition includes a probe set comprising or consisting of isolated polynucleotides that selectively hybridize to SEQ ID NO:9 (BIRC5), or complements thereof. In these various preferred embodiments of HR− composition 3, it is further preferred that the different probe sets in total selectively hybridize to at least 45%, 50%, 55%, 60%, 65%, 70%, 80%, 85%, 90%, 95%, or 100% of the probe sets comprise or consist of one or more isolated polynucleotides that selectively hybridize to one of these 3 recited nucleic.

The compositions of this third aspect of the invention are especially preferred for use in RNA expression analysis from the genes in a tissue of interest, such as breast tissue samples (including but not limited to biopsies, lumpectomy samples, and solid tumor samples), fibroids, circulating tumor cells that have been shed from a tumor, blood samples (such as blood smears), and bone marrow cells. Such polynucleotides according to this aspect of the invention can be of any length that permits selective hybridization to the nucleic acid of interest. In various preferred embodiments of the third aspect of the invention and related aspects and embodiments disclosed below, the isolated polynucleotides comprise or consist of at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 nucleotides according to a nucleic acid selected from the group consisting of SEQ ID NO:1-17, or complements thereof. In further embodiments, an isolated polynucleotide according to this third aspect of the invention comprise or consist of a nucleic acid according to one of SEQ ID NO:1-17, or complements thereof.

The compositions of the various aspects and embodiments of the invention can be in lyophilized form, or preferably comprise a solution containing the isolated polynucleotides, including but not limited to buffer solutions, hybridization solutions, and solutions for keeping the compositions in storage. Such a solution can be made as such, or the composition can be prepared at the time of hybridizing the polynucleotides to a target sequence, as discussed below.

Alternatively, the compositions can be placed on a solid support, such as in a microarray, bead, or microplate format. The term “microarray” as used herein refers to a plurality of probe sets immobilized on a solid surface to which sample nucleic acids are hybridized (such as breast cancer mRNA or derived cDNA).

Thus, in a fourth aspect, the present invention provides microarrays comprising a support structure on which are arrayed one or more probe sets according to the compositions of the invention, as disclosed above. In this aspect, a single probe set can be present at a single location on the array, or different polynucleotides from a single probe set can be present at different and defined locations on the array.

In this aspect, the polynucleotides are immobilized on a microarray solid surface. Other nucleic acids, such as reference or control nucleic acids, can be optionally immobilized on the solid surface as well. Methods for immobilizing nucleic acids on a variety of solid surfaces are well known to those of skill in the art. A wide variety of materials can be used for the solid surface. Examples of such solid surface materials include, but are not limited to, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose and polyacrylamides.

A variety of different materials may be used to prepare the microarray solid surface to obtain various properties. For example, proteins (e.g., bovine serum albumin) or mixtures of macromolecules (e.g., Denhardt's solution) can be used to minimize non-specific binding, simplify covalent conjugation, and/or enhance signal detection. If covalent bonding between a compound and the surface is desired, the surface will usually be functionalized or capable of being functionalized. Functional groups which may be present on the surface and used for linking include, but are not limited to, carboxylic acids, aldehydes, amino groups, cyano groups, ethylenic groups, hydroxyl groups, and mercapto groups. Methods for linking a wide variety of compounds to various solid surfaces are well known to those of skill in the art.

In a preferred embodiment of this fourth aspect, the locations on the array containing probe sets of the present invention range in size between 1 μm and 1 cm in diameter, more preferably between 1 μm and 5 mm in diameter, and even more preferably between 5 μm and 1 mm in diameter. The polynucleotides of the probe sets may be arranged on the solid surface at different densities, depending on factors such as the nature of the label, the solid support, and the size of the polynucleotide. One of skill will recognize that each location on the microarray may comprise a mixture of polynucleotides of different lengths and sequences from a given probe set. The length and complexity of the polynucleotides fixed onto the locations can be adjusted to provide optimum hybridization and signal production for a given hybridization procedure, and to provide the required resolution.

In a fifth aspect, the present invention provides methods for classifying a breast tumor, comprising

(a) contacting a nucleic acid sample obtained from a subject having a breast tumor with polynucleotide probes that, in total, selectively hybridize to two or more genomic regions selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the contacting occurs under conditions to promote selective hybridization of the polynucleotides of the probe set to the two or more genomic regions;

(b) detecting formation of hybridization complexes;

(c) determining whether one or more of the genomic regions are present in an altered copy number in the nucleic acid sample; and

(d) correlating an altered copy number of one or more of the genomic regions with a breast cancer classification.

The nucleic acid sample used in the methods of the present invention can be from any source useful in classifying a breast tumor, including but not limited to breast tissue samples (including but not limited to biopsies, lumpectomy samples, and solid tumor samples), fibroids, circulating tumor cells that have been shed from a tumor, blood samples (such as blood smears), and bone marrow cells. The nucleic acid sample is preferably a cellular DNA sample. In a preferred embodiment, the nucleic acid sample is a human nucleic acid sample.

In the fifth aspect of the invention the methods are used to detect genomic amplifications or deletions associated with breast cancer. As used herein “associated with breast cancer” means that an altered copy number of one or more of these genomic regions can be used to classify a feature of the breast tumor or the prognosis of a patient from whom the nucleic acid sample was taken, including the following:
(a) Diagnosis of breast cancer (benign vs. malignant tumor);
(b) Metastatic potential, potential to metastasize to specific organs, or course of the tumor;
(c) Stage of the tumor;
(d) Patient prognosis in the absence of chemotherapy or hormonal therapy;
(e) Prognosis of patient response to treatment (chemotherapy, radiation therapy, and/or surgery to excise tumor)
(f) Predicted optimal course of treatment for the patient;
(g) Prognosis for patient relapse after treatment; and
(h) Patient life expectancy.

Thus, the methods of this aspect of the invention provide information on, for example, breast cancer diagnosis, and patient prognosis in the presence or absence of chemotherapy, a predicted optimal course for treatment of the patient, and patient life expectancy. In a preferred embodiment, the breast cancer classification comprises a prognosis of the recurrence of the breast tumor. In a further preferred embodiment, an alteration (ie: increase or decrease) in the copy number of the one or more genomic regions is correlated with an increased risk of recurrence of the breast tumor. In a further preferred embodiment, alterations in the normal expression levels of the one or more nucleic acid targets is correlated with a higher risk of recurrence of the breast tumor.

As used herein for all aspects and embodiments of the methods, an “alteration in copy number means any increase or decrease in copy number of the genomic region or target relative to the copy number in a normal diploid human genome. It is understand that for most expressed genes in the human genome this normal number will be two.

One skilled in the art will further understand that “alteration in the expression levels” (as discussed below) means any deviation from the level of expression relative to the same normal healthy tissue. It is further understood that “increased risk” means to be at a higher risk relative to all others having similar or identical clinical and/or pathological characteristics, in the absence of the information obtained using the markers as described herein. As used herein, “recurrence” means tumor local recurrence (including ipsilateral, local, or contralateral), metastasis, or death from breast cancer.

In a preferred embodiment, the determining in step (c) comprises determining whether 2, 3, 4, 5, 6, 7, 8, 9, 10, 11, 12, 13, or 14 of the recited genomic regions are present in an altered copy number, wherein such altered copy number correlates with a particular breast cancer classification, preferably a classification that the breast cancer is likely to recur.

Thus, the invention further provides methods for making a treatment decision for a breast cancer patient, comprising carrying out the methods for classifying a breast tumor according to the different aspects and embodiments of the present invention, and then weighing the results in light of other known clinical and pathological risk factors, in determining a course of treatment for the breast cancer patient.

For example, a patient that is shown by the methods of the invention to have an increased risk of recurrence could be treated more aggressively with standard therapies, such as chemotherapy, radiation therapy, and/or mastectomy, or novel or experimental therapies under clinical investigation.

In various preferred embodiments of the methods of the fifth aspect of the invention, the polynucleotide probes comprise compositions selected from the various aspects and embodiments of the compositions of the invention disclosed above. In a most preferred embodiment, the polynucleotides probes comprise a detectable label, as disclosed above, and in particular the different probe sets of the compositions of the invention comprise distinguishable detectable labels, to facilitate analysis of which genomic region(s) is/are the site of the an altered copy number.

In various other preferred embodiments of the methods of the invention, the compositions for use in the methods, in total, selectively hybridize to three, four, five, six, seven, eight, nine, ten, eleven, twelve, thirteen, or fourteen genomic regions selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the contacting occurs under conditions to promote selective hybridization of the one or more probe sets to the three, four, five, six, seven, eight, nine, ten, eleven, twelve, thirteen, or fourteen genomic regions.

In various further preferred embodiments of the methods of this fifth aspect of the invention, the compositions comprise or consist of one or more of the HR+ and HR− compositions of the invention, and the methods are used to provide a classification of HR+ and/or HR− breast tumors. In a preferred embodiment, the classification comprises prognosing a recurrence of the HR+ or HR− breast tumors. Thus, in one preferred embodiment of the fifth aspect of the invention, the breast cancer biomarker comprises or consists of between 2 and 35 different probe sets, wherein the different probe sets comprise or consist of isolated polynucleotides that in total selectively hybridize to at least the following genomic regions: 20q13.2 (includes CYP24) and 3p23 (includes PDCD6IP). This embodiment (“HR+ composition 1”) is demonstrated herein to be particularly effective for classifying hormone receptor positive breast tumors, where “hormone receptor positive” is defined as positive for either or both of estrogen receptors and progesterone receptors. In this embodiment, it is further preferred that the HR+ composition comprises isolated polynucleotides in total also selectively hybridize to genomic region 17q25.3 (includes BIRC5). In various further preferred embodiments of the HR+ composition, at least 45%, 50%, 55%, 60%, 65%, 70%, 80%, 85%, 90%, 95%, or 100% of the probe sets comprise or consist of one or more isolated polynucleotides that selectively hybridize to one of these 2 or 3 recited genomic regions.

In a further preferred embodiment of the fifth aspect of the invention, the polynucleotide probes comprise or consist of a breast cancer biomarker that comprises or consists of between 2 and 35 different probe sets, and wherein the different probe sets in total selectively hybridize to at least the following genomic regions: 17q21.2 (includes SMARCE 1) and 17q21.1 (includes NR1D1). This embodiment (“HR− composition 1”) is demonstrated herein to be particularly effective for classifying hormone receptor negative breast tumors, where “hormone receptor negative” is defined as negative for either or both of estrogen receptors and progesterone receptors. In this embodiment, it is further preferred that the polynucleotides in total selectively hybridize to genomic region 17q25.3 (includes BIRC5). In these various HR− composition embodiments, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of these 2 or 3 recited genomic regions.

In various further embodiments of the methods of the invention, the compositions for use in the methods comprise a breast cancer biomarker comprising or consisting of between 2 and 42 different probe sets, wherein at least 40% of the different probe sets comprise one or more isolated polynucleotides that selectively hybridize to a nucleic acid according to formula 1, or complements thereof:

X1-X2-X3;

wherein X2 is a human genomic insert contained within a bacterial artificial chromosome (“BAC”) selected from the group consisting of SEQ ID NOS: 18-65 (see FIG. 1), wherein X1 and X3 are independently 0-500 kB of human genomic nucleic acids flanking X2 in the human genome; and

wherein the different polynucleotide probe sets in total selectively hybridize to at least two non-overlapping genomic sequences according to formula 1. Such biomarkers may preferably consist of three, four, five, six, seven, eight, nine, ten, eleven, twelve, thirteen, fourteen, fifteen, sixteen, or seventeen different probe sets that selectively hybridize to a nucleic acid sequence according to formula 1, or its complement. In each of these embodiments, it is further preferred that at least 45%, 50%, 55%, 60%, 65%, 70%, 80%, 85%, 90%, 95%, or 100% of the probe sets for a given breast cancer biomarker comprise or consist of one or more isolated polynucleotides that selectively hybridize to a nucleic acid sequence according to formula 1, or complements thereof, wherein the different polynucleotide probe sets in total selectively hybridize to at least three, four, five, six, seven, eight, nine, ten, eleven, twelve, thirteen, fourteen, fifteen, sixteen, or seventeen non-overlapping genomic sequences according to formula 1. It is further preferred that X1 and X3 are 0-400 kb; 0-300 kb; 0-200 kb; 0-100 kb; or 0 kb. It is further preferred that the different probe sets of a breast cancer biomarker comprise or consist of one or more polynucleotide sequences of at least 10 nucleotides of a nucleic acid according to formula 1, or complements thereof. In a further preferred embodiment, the different probe sets of a breast cancer biomarker comprise or consist of one or more polynucleotides of at least 10 nucleotides of a nucleic acid selected from the group consisting of SEQ ID NOS:18 to 511, or complements thereof.

In a further preferred embodiment of the methods of the invention, the different probe sets in total selectively hybridize to at least two different nucleic acids according to Formula I having X2 groups as follows:

a) one or more of SEQ ID NO:62-65 (includes CYP24), or complements thereof; and

b) one or more of SEQ ID NO:44-46 (includes PDCD6IP)), or complements thereof.

This embodiment (“HR+ composition 2”) is demonstrated herein to be particularly effective for classifying hormone receptor positive breast tumors, where “hormone receptor positive” is defined as positive for either or both of estrogen receptors and progesterone receptors. In a further embodiment of the HR+ composition 2, the different probe sets in total selectively hybridize to one or more of SEQ ID NO:41-43 (includes BIRC5). In various further preferred embodiments, the different probe sets in total selectively hybridize to one or more nucleic acid from each of the following groups:

(a) SEQ ID NOS: 256-327 (unique sequence probes from the CYP24-containing BAC)), or complements thereof; and

(b) SEQ ID NOS: 160-255 (unique sequence probes from the PDCD6IP-containing BAC)), or complements thereof.

Nucleic acids in group (a) are unique sequence regions from the BAC including CYP24, and nucleic acids in group (b) are unique sequence regions from the BAC including PDCD6IP. Thus, this embodiment of HR+ composition 2 provides unique sequence probes for use if the methods of the invention, which obviates the need for competitor DNA in hybridization assays. In a further embodiment, the unique sequence probes for HR+ composition 2 further include different probe sets that selectively hybridize to one or more nucleic acid from the groups:

(c) SEQ ID NOS:416-511 (unique sequence probes from the BIRC5-containing BAC)), or complements thereof.

In these various embodiments of HR+ composition 2, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of the 2 or 3 recited nucleic acids.

In a further preferred embodiment of the fifth aspect of the invention, the different probe sets in total selectively hybridize to at least two different nucleic acids according to Formula I having X2 groups as follows:
a) one or more of SEQ ID NO:57-59 (includes NR1D1), or complements thereof; and
b) SEQ ID NO:40 (includes SMARCE1), or complements thereof.

This embodiment (“HR− composition 2”) is demonstrated herein to be particularly effective for classifying hormone receptor negative breast tumors, where “hormone receptor negative” is defined as negative for either or both of estrogen receptors and progesterone receptors. In a further embodiment of the HR− composition 2, the different probe sets in total selectively hybridize to one or more of SEQ ID NO:41-43 (includes BIRC5), or complements thereof. In various further preferred embodiments of HR− composition 2, the different probe sets in total selectively hybridize to one or more nucleic acid from each of the following groups:

(a) SEQ ID NOS:328-415 (unique sequence probes from the NR1D1-containing BAC), or complements thereof; and

(b) SEQ ID NOS:66-159 (unique sequence probes from the SMARCE-containing BAC), or complements thereof.

Nucleic acids in group (a) are unique sequence regions from the BAC including NR1D1, and nucleic acids in group (b) are unique sequence regions from the BAC including SMARCE. Thus, this embodiment of HR-composition 2 provides unique sequence probes for use if the methods of the invention, which obviates the need for competitor DNA in hybridization assays. In a further embodiment, the unique sequence probes for HR− composition 2 include probe sets comprising or consisting of polynucleotides that selectively hybridize to one or more nucleic acid from the groups:

(c) SEQ ID NOS:416-511 (unique sequence probes from the BIRC5-containing BAC), or complements thereof.

In these embodiments of HR− composition 2, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of these 3 recited nucleic acids.

In various further preferred embodiments of the methods of the invention, the polynucleotides in the probe set independently comprise or consist of at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10,000; 15,000; 20,000; 25,000; 30,000; 35,000; 40,0000; 45,000; 50,000; 60,000; 70,000; 80,000; 90,000; 100,000; 110,000; 120,000; 130,000; 140,000; 150,000; 160,000; 170,000; 180,000; 190,000; 200,000; 210,000; or 220,000 nucleotides of the relevant sequence.

In various further embodiments of the methods of the invention, the compositions comprise a breast cancer biomarker comprising or consisting of between 2 and 42 different probe sets, wherein at least 40% of the different probe sets comprise one or more isolated polynucleotides that selectively hybridize to a nucleic acid according to one of SEQ ID NO:1-17 or complements thereof; wherein the different probe sets in total selectively hybridize to at least two of the recited nucleic acids according to SEQ ID NO:1-17 or complements thereof. In various preferred embodiments, the composition comprises a breast cancer biomarker consisting of three, four, five, six, seven, eight, nine, ten, eleven, twelve, thirteen, fourteen, fifteen, sixteen, or seventeen different probe sets that selectively hybridize to a nucleic acid according to one of SEQ ID NO:1-17 or complements thereof, wherein the different probe sets in total selectively hybridize to at least three, four, five, six, seven, eight, nine, ten, eleven, twelve, thirteen, fourteen, fifteen, sixteen, or seventeen of the recited nucleic acids according to SEQ ID NO:1-17 or complements thereof. In each of these embodiments, it is further preferred that at least 45%, 50%, 55%, 60%, 65%, 70%, 80%, 85%, 90%, 95%, or 100% of the probe sets for a given breast cancer biomarker comprise or consist of one or more isolated polynucleotides that selectively hybridize to a nucleic acid according to SEQ ID NO:1-17, or complements thereof. In further preferred embodiments, the different probe sets of a breast cancer biomarker comprise or consist of one or more polynucleotides of at least 10 nucleotides of a nucleic acid according to SEQ ID NO:1-17, or complements thereof. In various further preferred embodiments, the isolated polynucleotides comprise or consist of at least 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 nucleotides according to a nucleic acid selected from the group consisting of SEQ ID NO:1-17, or complements thereof. In further embodiments, the isolated polynucleotide comprises or consists of a nucleic acid according to one of SEQ ID NO:1-17, or complements thereof.

In a preferred embodiment of each embodiment of the fifth aspect of the invention, the method comprises calculating a prognostic index, by employing a linear combination of the log(target copy numbers) with coefficients computed from a logistic regression analysis. Based on the value of the prognostic index, the samples are categorized into appropriate risk groups, for example, low, moderate, and high. For example, a prognostic index for a 3-probe set breast cancer biomarker can take the form of:

PI(prognostic index)=a1*log(copy number gene 1)+a2*log(copy number gene 2)+a3*log(copy number gene 3).

For example, suppose that through a prior analysis, the coefficients were found to be:

a1=1,

a2=1,

a3==2,

And that a PI<1 was found to correspond to a low risk of recurrence.

For a breast cancer patient with a tumor having the following characteristics:

Copy number gene 1=4.

Copy number gene 2=2.

Copy number gene 3=4.

The PI is computed as:

PI=1*log(4)+1*log(2)−2*log(4)=−0.693<1,

so this patient would be considered at low risk of distant recurrence.

Let CNi denote the copy number for gene i. Note that the PI may be written as PI=log((CN1̂a1)*(CN2̂a2)*(CN3̂a3)). Given that PI is predictive of recurrence, then so is exp(PI), and thus we can define a new prognostic index as:

PI=(CN1̂a1)*(CN2̂a2)*(CN3̂a3).

If there are both positive and negative coefficients, then this PI can be expressed as a ratio. For the example coefficients above:

PI=CN1*CN2/CN3̂2.

In another example, the prognostic index can be a ratio of gene copy numbers, such as:

Hormone +: PI=CYP24*BIRC5/(PDCD6IP²), where the gene name denotes the copy number of that gene. For example, suppose that through a prior analysis PI<1 was found to correspond to a low risk of recurrence. For a breast cancer patient with a tumor having the following characteristics:

CYP24=4.

BIRC5=4.

PDCD6IP=2.

The PI is computed as: PI=4*4/(2̂2)=4>1, so this patient would be considered at a higher risk of distant recurrence.

Hormone −: PI=NR1D1²/(BIRC5*SMARCE1), where the gene name denotes the copy number of that gene. For example, suppose that through a prior analysis PI<(1/2) was found to correspond to a low risk of recurrence. For a breast cancer patient with a tumor having the following characteristics:

SMARCE1=4.

NR1D1=2.

BIRC5=4.

The PI is computed as: PI=(2̂2)/(4*4)=1/4<1/2, so this patient would be considered at a low risk of distant recurrence.

For test implementation, ratio predictors have certain advantages over linear combination predictors. The above ratio PI's are unit-less: any bias in gene copy measurements will tend to cancel out.

Any conditions, including hybridization reagents and wash conditions to remove unbound probe, in which the nucleic acid probes bind selectively to the target in the nucleic acid sample to form a hybridization complex, and minimally or not at all to other sequences, can be used in the methods of the present invention, as discussed above. Further optional steps can include, but are not limited to, pre-hybridization of the nucleic acid sample and use of competitor nucleic acids.

Any method for detecting formation of hybridization complexes and determining an alteration in gene copy number can be used, including but not limited to in situ hybridization (such as fluorescent in situ hybridization (FISH)), polymerase chain reaction (PCR) analysis, reverse transcription polymerase chain reaction (RT-PCR) analysis, Southern blotting, Northern blotting, array-based methods, and/or comparative genomic hybridization.

In a preferred embodiment, detection is performed by in situ hybridization (“ISH”). In situ hybridization assays are well known to those of skill in the art. Generally, in situ hybridization comprises the following major steps (see, for example, U.S. Pat. No. 6,664,057): (1) fixation of tissue, biological structure, or nucleic acid sample to be analyzed; (2) pre-hybridization treatment of the tissue, biological structure, or nucleic acid sample to increase accessibility of the nucleic acid sample (within the tissue or biological structure in those embodiments), and to reduce nonspecific binding; (3) hybridization of the probe to the nucleic acid sample; (4) post-hybridization washes to remove probe not bound in the hybridization and (5) detection of hybridization complexes. The reagent used in each of these steps and their conditions for use varies depending on the particular application. In a particularly preferred embodiment, ISH is conducted according to methods disclosed in U.S. Pat. Nos. 5,750,340 and/or 6,022,689, incorporated by reference herein in their entirety.

In a typical in situ hybridization assay, cells are fixed to a solid support, typically a glass slide. The cells are typically denatured with heat or alkali and then contacted with a hybridization solution to permit annealing of labeled probes specific to the target nucleic acid sequence. The polynucleotides of the invention are typically labeled, as discussed above. In some applications it is necessary to block the hybridization capacity of repetitive sequences. In this case, human genomic DNA or Cot-1 DNA is used to block non-specific hybridization.

In a further embodiment, an array-based format can be used in which the polynucleotides of the invention can be arrayed on a surface and the human nucleic sample is hybridized to the polynucleotides on the surface. In this type of format, large number of different hybridization reactions can be run essentially “in parallel.” This provides rapid, essentially simultaneous, evaluation of a large number of nucleic acid probes. Methods of performing hybridization reactions in array based formats are also described in, for example, Pastinen (1997) Genome Res. 7:606-614; (1997) Jackson (1996) Nature Biotechnology 14:1685; Chee (1995) Science 274:610; WO 96/17958. Methods for immobilizing the polynucleotides on the surface and derivatizing the surface are known in the art; see, for example, U.S. Pat. No. 6,664,057, and are also described above.

In a sixth aspect, the present invention provides methods for classifying a breast tumor comprising:

(a) contacting a mRNA-derived nucleic acid sample obtained from a subject having a breast tumor with nucleic acid probes that, in total, selectively hybridize to two or more nucleic acid targets selected from the group consisting of SEQ ID NO:1-17 or complements thereof; wherein the contacting occurs under conditions to promote selective hybridization of the nucleic acid probes to the nucleic acid targets, or complements thereof, present in the nucleic acid sample;

(b) detecting formation of hybridization complexes between the nucleic acid probes to the nucleic acid targets, or complements thereof, wherein a number of such hybridization complexes provides a measure of gene expression of the one or more nucleic acids according to SEQ ID NO:1-17; and

(c) correlating an alteration in gene expression (ie, an increase or decrease) of the one or more nucleic acids according to SEQ ID NO:1-17 relative to control with a breast cancer classification. In a preferred embodiment, the classification comprises breast cancer recurrence.

The method according to the sixth aspect of the invention detects alterations in gene expression of one or more of the markers according to SEQ ID NO:1-17 relative to a control with a modification in expression relative to control correlating with a classification of the breast tumor as likely to recur.

Any control known in the art can be used in the methods of the invention. For example, the expression level of a gene known to be expressed at a relatively constant level in both cancerous and non-cancerous tumor tissue can be used for comparison. Alternatively, the expression level of the genes targeted by the probes can be analyzed in non-cancerous RNA samples equivalent to the test sample. Those of skill in the art will recognize that many such controls can be used in the methods of the invention.

In the sixth aspect of the invention the methods are used to detect gene expression alterations associated with breast cancer. As used herein “associated with breast cancer” means that an altered expression level of one or more of the markers can be used to classify a feature of the breast tumor or the prognosis of a patient from whom the nucleic acid sample was taken, including the following:
(a) Diagnosis of breast cancer (benign vs. malignant tumor);
(b) Metastatic potential, potential to metastasize to specific organs, or course of the tumor;
(c) Stage of the tumor;
(d) Patient prognosis in the absence of chemotherapy or hormonal therapy;
(e) Prognosis of patient response to treatment (chemotherapy, radiation therapy, and/or surgery to excise tumor)
(f) Predicted optimal course of treatment for the patient;
(g) Prognosis for patient relapse after treatment; and
(h) Patient life expectancy.

Thus, the methods of this aspect of the invention provide information on, for example, breast cancer diagnosis, and patient prognosis in the presence or absence of chemotherapy, a predicted optimal course for treatment of the patient, and patient life expectancy. In a preferred embodiment, the breast cancer classification comprises a prognosis of the recurrence of the breast tumor. In a further preferred embodiment, an altered expression level of the one or more nucleic acid targets is correlated with an increased recurrence rate of the breast tumor compared to control. As used herein, “recurrence” means tumor local recurrence (including ipsilateral, local, or contralateral), metastasis, or death from breast cancer.

In a further preferred embodiment, alterations in the normal expression levels of the one or more nucleic acid targets are correlated with a higher risk of recurrence of the breast tumor. One skilled in the art will understand that “alteration in the expression levels” means any deviation from the level of expression relative to the same normal healthy tissue. It is further understood that “increased risk” means to be at a higher risk relative to all others having similar or identical clinical and/or pathological characteristics, in the absence of the information obtained using the markers as described herein.

As used herein for all aspects and embodiments of the method, an alteration (ie: an increase or decrease) in gene expression relative to control is any increase or decrease relative to normal tissue counterpart of the disease state. In various embodiments, the increase or decrease is at least a 10%, 20%, 30%, 40%, 50%, 60%, 70%, 80%, 90%, 100%, 200%, or greater increase or decrease.

Thus, the invention further provides methods for making a treatment decision for a breast cancer patient, comprising carrying out the methods for classifying a breast tumor according to the different aspects and embodiments of the present invention, and then weighing the results in light of other known clinical and pathological risk factors, in determining a course of treatment for the breast cancer patient. For example, a patient that is shown by the methods of the invention to have an increased risk of recurrence could be treated more aggressively with standard therapies, such as chemotherapy, radiation therapy, and/or surgical removal of the tumor.

The mRNA-derived nucleic acid sample used in the methods of the present invention can be mRNA or cDNA derived from the mRNA. The RNA sample used in the methods of the present invention can be from any source useful in classifying a breast tumor, including but not limited to breast tissue samples, fibroids, and blood samples including bone marrow cells. In a preferred embodiment, the RNA sample is a human RNA sample. The nucleic acid sample is preferably a human cellular DNA or RNA sample, such as a sample prepared for in situ hybridization.

In various preferred embodiments of the methods of the sixth aspect of the invention, the nucleic acid probes are selected from the various aspects and embodiments of the compositions disclosed above, particularly the third aspect of the invention and preferred embodiments thereof. In a most preferred embodiment, the polynucleotides of the probe sets comprise a detectable label, as disclosed above, and in particular the different probe sets comprise distinguishable detectable labels, to facilitate analysis of gene expression of multiple targets.

In various other preferred embodiments, the nucleic acid probes in total selectively hybridize to three, four, five, six, seven, eight, nine, ten, eleven, twelve, thirteen, fourteen, fifteen, sixteen, or seventeen different nucleic acids according to SEQ ID NO:1-17 or complements thereof, and the alteration in gene expression of two, three, four, five, six, seven, eight, nine, ten, eleven, twelve, thirteen, fourteen, fifteen, sixteen, or seventeen of the nucleic acids according to SEQ ID NO:1-17, or complements thereof, are correlated with a breast cancer classification, preferably with recurrence.

In a further preferred embodiment of the sixth aspect of the invention, the composition for use comprises different probe sets that in total selectively hybridize to at least SEQ ID NO:17 (CYP24), and SEQ ID NO:10 (PDCD6IP), or complements thereof. This embodiment (“HR+ composition 3”) is demonstrated herein to be particularly effective for classifying hormone receptor positive breast tumors, where “hormone receptor positive” is defined as positive for either or both of estrogen receptors and progesterone receptors. In this embodiments of HR+ composition 3, it is further preferred that the different probe sets in total selectively hybridize to SEQ ID NO:9 (BIRC5). In these various embodiments of HR+ composition 3, it is further preferred that at least 45%, 50%, 55%, 60%, 65%, 70%, 80%, 85%, 90%, 95%, or 100% of the probe sets comprise or consist of one or more isolated polynucleotides that selectively hybridize to one of these 2 or 3 recited nucleic acids.

In a further preferred embodiment of the sixth aspect of the invention, the composition for use comprises different probe sets that in total selectively hybridize to at least SEQ ID NO:15 (NR1D1) and SEQ ID NO:8 (SMARCE1), or complements thereof. This embodiment (“HR− composition 3”) is demonstrated herein to be particularly effective for classifying hormone receptor negative breast tumors, where “hormone receptor negative” is defined as negative for either or both of estrogen receptors and progesterone receptors. In this embodiment of HR− composition 3, it is further preferred that the different probe sets in total selectively hybridize to SEQ ID NO:9 (BIRC5). In these various preferred embodiments of HR− composition 3, it is further preferred that the different probe sets in total selectively hybridize to at least 45%, 50%, 55%, 60%, 65%, 70%, 80%, 85%, 90%, 95%, or 100% of the probe sets comprise or consist of one or more isolated polynucleotides that selectively hybridize to one of these 3 recited nucleic.

Such probes according to this aspect of the invention can be of any length that permit selective hybridization under stringent conditions to the nucleic acid of interest, and preferably are at least 10 nucleotides in length. In various further embodiments, the probes according to this embodiment are at least 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000 nucleotides in length. In a further embodiment, the probes according to this aspect of the invention are complementary to the entire recited nucleic acid. The probes of this embodiment may be RNA or DNA and may be single or double stranded.

In a most preferred embodiment of this aspect, the nucleic acid probes comprise or consist of single stranded anti-sense polynucleotides of the nucleic acid compositions of the invention. For example, in mRNA fluorescence in situ hybridization (FISH) (ie. FISH to detect messenger RNA), only an anti-sense probe strand hybridizes to the single stranded mRNA in the RNA sample, and in that embodiment, the “sense” strand oligonucleotide can be used as a negative control.

Alternatively, DNA probes can be used as probes, preferably those according to the compositions of the invention. In this embodiment, it is preferable to distinguish between hybridization to cytoplasmic RNA and hybridization to nuclear DNA. There are two major criteria for making this distinction: (1) copy number differences between the types of targets (hundreds to thousands of copies of RNA vs. two copies of DNA) which will normally create significant differences in signal intensities and (2) clear morphological distinction between the cytoplasm (where hybridization to RNA targets would occur) and the nucleus will make signal location unambiguous. Thus, when using double stranded DNA probes, it is preferred that the method further comprises distinguishing the cytoplasm and nucleus in cells being analyzed within the bodily fluid sample. Such distinguishing can be accomplished by any means known in the art, such as by using a nuclear stain such as Hoechst 33342, or DAPI which delineate the nuclear DNA in the cells being analyzed. In this embodiment, it is preferred that the nuclear stain is distinguishable from the detectable probes. It is further preferred that the nuclear membrane be maintained, i.e., that all the Hoechst or DAPI stain be maintained in the visible structure of the nucleus.

Hybridization conditions and other details of the methods of this aspect are as described above for altered copy number analysis. In a preferred embodiment, RNA FISH is employed using standard methods in the art.

In each of the above aspects and embodiments, detection of hybridization is typically accomplished through the use of a detectable label on the nucleic acid probes, such as those described above. The label can be directly incorporated into the polynucleotide, or it can be attached to a molecule which hybridizes or binds to the polynucleotide. The labels may be coupled to the probes in a variety of means known to those of skill in the art, as described above. In a preferred embodiment, the detectable labels on the different probe sets of the compositions of the invention are distinguishable from each other, as discussed above. The label can be detected can be by any techniques, including but not limited to spectroscopic, photochemical, biochemical, immunochemical, physical or chemical techniques, as discussed above and below.

In a further aspect, the present invention provides kits for use in the methods of the invention, comprising the compositions of the invention and instructions for their use. In a preferred embodiment, the probe sets are labeled, preferably so as to distinguish different probe sets, as disclosed above. In a further preferred embodiment, the probe sets are provided in solution, most preferably in a hybridization buffer to be used in the methods of the invention. In a further embodiment, the probe sets are provided on a solid support, such as those described above. In further embodiments, the kit also comprises wash solutions and/or pre-hybridization solutions.

### EXAMPLES

### Example 1

Currently employed clinical and tumor pathology predictors of breast cancer prognosis are not very accurate. As a result, many more patients are subjected to adjuvant chemotherapy than will benefit from such treatment and many patients that will have a poor outcome are not identified early for aggressive treatment Van't Veer et al (2002) addressed the question of identifying a gene expression profile correlating with prognosis. The data collected by his group consisted of gene expression measurements across 24481 genes for 97 breast tumor samples with accompanying clinical data. Applying a univariate gene selection mechanism, they identified a group of 70 genes useful in predicting prognosis:

Van't Veer 70 gene marker

| | |
|---|---|
| Accuracy | 80.8% |
| Sensitivity | 91.2% |
| Specificity | 72.7% |

Accuracy is defined herein as the proportion of samples correctly classified by a biomarker. Sensitivity refers to the proportion of poor prognosis samples correctly classified as such, and specificity refers to the proportion of good prognosis samples correctly classified as such.

However, the clinical utility of a 70 gene marker is limited by the cost and complexity of coordinating 70 measurements. Additionally, the measurement of gene expression in solid tumor tissue samples requires tissue handling procedures not in common practice. For the clinic, an alternative implementation of the discovered molecular signatures is preferred. One such approach is FISH (fluorescence in situ hybridization) measurement of DNA aberrations. FISH is currently in wide clinical use for the characterization of a variety of tumor DNA aberrations including: chromosome aneuploidy, translocations, and gene amplification.

### Mapping the Molecular Signatures

It has recently been demonstrated by Pollack et al (2002) that, for tumors of the breast, for a significant proportion of the genome, gene expression is correlated with gene amplification. From DNA copy number and RNA gene expression measurements for 6095 genes across 37 tumor samples, Pollack et al found that 12% of all gene expression variation is directly attributable to gene amplification. Additionally, they found that for highly amplified regions, 62% of the genes located in these regions exhibit moderately or highly elevated expression. Thus, for those molecular signatures that map to regions of high correlation, a FISH-based assay is an alternative to gene expression measurement. In order to identify such molecular signatures, this data was analyzed concurrently with the above-described Van't Veer gene expression data.

From this concurrent analysis, we have identified nine genes whose RNA gene expression is prognostic of disease free survival, and additionally whose RNA expression is highly correlated with DNA copy number.

### Validation of Gene Selection in Independent Dataset

From a previous publication (Sorlie et al 2001) prognosis data, in the form of DFS (disease free survival), were available for a subset of the Pollack breast cancer DNA amplification data. This prognosis data was not used in the identification of the nine genes, and thus can be used to test the prognostic accuracy of the nine genes.

Of the 26 samples for which survival data are available, 18 were collected from patients with a poor prognosis (less than 5 years DFS), and 8 from patients with a good prognosis (greater than 5 years DFS). Due to the small number of samples for which DFS was available, we limited our analysis to one and two gene combinations from the previously discovered nine genes. We found that the most prognostic single gene marker (AL080059) is 73% accurate in predicting prognosis in the DNA amplification data. A combination of two (AL080059, ANXA11) of the top nine informative genes achieved the following performance on the independent prognosis data:

| | |
|---|---|
| Accuracy | 92.3% |
| Sensitivity | 94.4% |
| Specificity | 87.5% |

Listed below are 17 genes resulting from our concurrent analysis of gene expression, DNA amplification, and their prognostic utility in breast cancer. Included in this list are: the aforementioned 9 genes whose expression is highly correlated with amplification and also prognostic, an additional 2 genes with highly variable DNA copy number and prognostic gene expression, and an additional 6 genes determined directly from the DNA copy number data and Sorlie DFS data.

TABLE 3
Breast Cancer Prognosis Genes

| NAME | GenBank Accession | Method of discovery |
|---|---|---|
| noname | AL080059 | dna/rna correlation |
| stk3 | NM_006281 | dna/rna correlation |
| ext1 | NM_000127 | dna/rna correlation |
| rad21 | NM_006265 | dna/rna correlation |
| anxa11 | NM_001157 | dna/rna correlation |
| fanca | NM_000135 | dna/rna correlation |
| znf144 | NM_007144 | dna/rna correlation |
| smarce | NM_003079 | dna/rna correlation |
| birc5 | NM_001168 | dna/rna correlation |
| pdcd6ip | NM_013374 | dna amplification |
| grb7 | NM_005310 | highly variable copy number |
| mln64 | NM_006804 | highly variable copy number |
| cyp24 | NM_000782 | dna amplification |
| impa1 | NM_005536 | dna amplification |
| hepsin | NM_002151 | dna amplification |
| nr1d1 | X72631 | dna amplification |
| znf207 | NM_003457 | dna amplification |

Thus, each of these genes can be used in marker sets for genomic DNA amplification associated with breast cancer.
van't Veer, L. J. et al. Gene expression profiling predicts clinical outcome of breast cancer. Nature. 2002 (415):530-536
Pollack, J. R. et al. Microarray analysis reveals a major direct role of DNA copy number alteration in the transcriptional program of human breast tumors. Proc Natl Acad Sci USA 2002 Oct. 1; 99(20):12963-8
Sorlie, T. et al. Gene expression patterns of breast carcinomas distinguish tumor subclasses with clinical implications. Proc Natl Acad Sci USA. 2001 Sep. 11; 98(19):10869-74.

### Example 2

### Validation of Multiplex Genomic Markers for Predicting Breast Cancer Recurrence in a FISH Assay Format

Predicting risk of recurrence in breast cancer patients is currently limited, resulting in the possibility of unnecessary adjuvant chemotherapy for some women and difficulty identifying those who could benefit from more aggressive treatment. Subsets of gene markers whose copy numbers are predictive of recurrence in breast cancer are described above. To validate the prognostic value of these patterns, we correlated copy number with recurrence using combinations of subsets of these markers in surgical specimens from women with invasive breast cancer. We measured the copy number of 17 candidate genomic markers, using fluorescent in situ hybridization (FISH) assays of paraffin embedded surgical specimens from 229 patients with early stage invasive cancer and known clinical outcomes with mean follow-up of 8.9 years. Univariate analysis showed that DNA copy number of 11 of the 17 candidate genomic markers in tumors was predictive of distant recurrence (P<0.001 to <0.03). We also identified predictive subsets of the 17 genomic markers.

### Study Population

Medical records from 723 cases of breast cancer diagnosed at the University of New Mexico Hospital between 1986 and 1999 were reviewed and 308 eligible cases were identified. Eligibility criteria included a diagnosis of Stage I, II or III invasive ductal carcinoma, availability of original clinical and pathological records, availability of archived tumor specimen, and at least four years of clinical follow-up. Mean follow-up was 8.9 years. Recurrence was defined as clinical evidence of metastasis or death from breast cancer. The study population consisted of 98 Stage I (33%), 118 Stage II (38%), and 61 Stage III (20%) patients. Treatments included mastectomy (59%), mastectomy/radiation (14%), lumpectomy (4%) and lumpectomy/radiation (21%). (Six patients underwent biopsy only biopsy only.) Patients received adjuvant hormone treatment (25%), chemotherapy (29%), chemotherapy and hormone treatment (15%) or no adjuvant treatment (22%). Tumors were classified as hormone receptor positive (HR+) if positive for either (or both) estrogen or progesterone receptors. Two hundred and nine (209) cases (68%) were HR+ and 83 (27%) were HR−. (Hormone status was undetermined in 16 cases, 5%).

Factors found to be significant predictors of recurrence included: younger age (P=4.3×10-4), pathological stage greater than pT1 (P=1.0×10-5), overall stage greater than I (P=6.0×10-6) and node status greater than N0 (P=4.2×10-8).

Neither hormone status (P=0.3208) nor ethnic origin (Caucasian vs. other, Hispanic vs. others, and Non-Caucasian vs others, P=>0.75) were found to be significant predictors of recurrence. Among the node negative group, neither age (P=0.4485) nor tumor size (P=0.1996) were found to be predictors of recurrence.

Pathology reports and microscopic slides were reviewed and diagnostic blocks selected for sectioning for FISH studies. FISH data were collected on 265 cases. The hybridizations were performed on serial sections and the personnel performing the hybridizations and signal collection were blinded to both the probe identities and clinical histories. The genomic probes used in this study were selected from the human “32K” BAC Re-Array (Children's Hospital Oakland Research Institute, http://bacpac.chori.org/). BAC clone inserts were verified by PCR and the chromosome localization verified by FISH hybridization to mitotic chromosomes. The 17 BAC probes were hybridized in pairs (labeled with Spectrum Red or Spectrum Green fluorochrome), and hybridizations followed standard protocols for sample deparaffinization, hybridization, and wash. Hybridization signals were collected, imaged, stored, and analyzed using MetaCyte automated FISH analytical hardware and software (MetaSystems, Altlusscheim, Germany). Generally, twenty 40× fields of view with good signal quality were operator-selected for each probe pair, and then captured in Metacyte. Regions not containing carcinoma cells (e.g. stromal or infiltrating lymphocytic cells) were excluded from further analysis using a process we refer to as “Virtual Microdissection.” Typically FISH signals from at least one hundred cells (usually 200) from at least 2 fields of view (usually 5-6) were analyzed for each gene probe pair. A few specimens (>5%) yielded smaller numbers of analyzable cells. Signal count data were collected in a “tiling” pattern designed by MetaSystems to minimize the effects of non-uniform distribution of nuclei in thin sections. Data were then transferred in flat file format for computational analysis.

### Methods and Results—Data Analysis

Raw Data. Raw data (DNA copy number per tile) were normalized to copy number per nuclear equivalent volume (NEV) by dividing the observed FISH signals per nuclear DAPI-stained cross-sectional area by a computed cross-sectional area per nuclear equivalent volume (Pahlplatz et al, 1995). Of the 308 cases eligible for the study, FISH data were obtained from 265 specimens. Thirty-one (31) cases had been collected after neo-adjuvant therapy, and five specimens were associated with local recurrences. These 36 cases were set aside for further study and are excluded from the analysis reported here, leaving 229 cases for analysis. Mean numbers of DNA copy number per nuclear equivalent volume (NEV) across all specimens are summarized in the histogram below.

Univariate Analysis. For the 18 probes, univariate analysis revealed that 11 were significantly associated with distant recurrence at p<0.05 (Wilcoxon test). P values for the individual genes are reported in the table below. This result can be compared with the number of expected false positives for no association between copy number and recurrence. For 18 probes, on average less than one probe would be determined significant at p=0.05. Thus these results validate the techniques employed in the original selection of the probes. However, we do not find any individual probes sufficiently prognostic to be clinically viable. Combinations of probes are required.

Patterns of DNA copy number. Patterns of copy number measurements for subsets of the 17 unique genomic regions correlating with distant recurrence were evaluated with respect to the performance of each subset's prognostic index. The prognostic index maps a score calculated from the copy numbers of the predictive pattern to risk of recurrence. The search for predictive patterns was conducted in a randomly chosen training set in each analysis. The remaining samples were withheld as a blinded test set. The prognostic index scores were grouped in low, moderate and high risk categories, and negative and positive predictive values were calculated. Prognostic patterns were studied in Hormone Positive (HR+) and Hormone Negative (HR−) cases. The best prognostic pattern in each subgroup was further tested on the subset of cases that was lymph node negative (HR+, N—). The gene composition of the Hormone Receptor Positive marker (HR+marker) and the Hormone Receptor Negative marker (HR− marker) are presented below and in Table 4.

### Three Gene Combinations:

Two distinct best combinations of 3 genes were predictive of recurrence in breast cancer:
   One for hormone receptor positive, Stage I, Stage II, & Stage III cases (where hormone receptor positive is defined as positive for either or both of estrogen receptors and progesterone receptors) Genes are: CYP24, PDCD6IP, BIRC 5
      This marker is also predictive in a subset that is hormone positive and node negative.
   One for hormone negative cases (estrogen receptor and progesterone receptor negative) Genes are NLRD1, SMARCE1, BIRC 5
The “combination” is specified by a function that includes coefficients that weight the importance of each gene to the classifier.

### Two Gene Combinations:

For both the HR+ and HR-cases combinations of two of the three genes are also predictive of recurrence.
   For HR+:CYP 24, PDCD6IP
   For HR−:NRLD1, SMARCE 1
For the two genes, the “combination” may be specified by a function as in the case of the three gene combinations above or as a ratio, linear or non-linear, of the copy numbers of the two. When the amplification of the two genes is not correlated (i.e. the ratio is different form the number one) there is a high risk of recurrence.

TABLE 4

| Marker | Genes |
|---|---|
| Hormone Positive | CYP24, PDCD6IP, |
| (HR+ Marker) | BIRC5 |
| Hormone Negative | NR1D1, SMARCE1, |
| (HR− Marker) | BIRC5 |

### Summary of Data Analyses

For this investigation we developed an algorithm for ranking combinations based on their ability to categorize samples into two or more risk groups. The algorithm employs a linear combination of the log (copy numbers) with coefficients computed from a logistic regression analysis. We term this linear combination the prognostic index. From the value of the prognostic index, the samples are categorized into risk groups. An objective function, based primarily on the actual risk difference between assigned low-risk and high-risk groups, was used in a global search to rank combinations and identify predictive combinations.

In the search phase, only a ‘training’ subset (25%-50%) of the data was used. The remaining samples were blinded as to recurrence, and used for testing of the identified predictive combinations. We found that the top performing combinations, as determined with the training data, also performed well on the blinded test data.

DNA copy number of genomic markers at each of two trios of loci [NR1D1, SMARCE1 and BIRC5] and [CYP24, PDCD6IP and BIRC5] powerfully predicted recurrence in hormone receptor negative and positive cancers, respectively. Based on the patterns of genomic marker amplification, patients could be placed into low and high risk groups with recurrences of 9% and 58%, respectively, in hormone receptor negative tumors and 9% and 50%, respectively, in hormone receptor positive cancers. The equations derived from the logistic regression analyses are of the form:

PI(prognostic index)=a1*log(copy number gene 1)+a2*log(copy number gene 2)+a3*log(copy number gene 3).

For both the hormone positive and hormone negative combinations, we noted that the computed coefficients were such that a ratio of gene copy numbers might be as significant a predictor as the above. In particular, we found the following ratio-based PI's to be significant predictors in our samples:

Hormone +: PI=CYP24*BIRC5/(PDCD6IP̂2),

Hormone −: PI=NR1D1̂2/(BIRC5*SMARCE1)

where the gene name denotes the copy number of that gene.

For test implementation, ratio predictors have certain advantages over linear combination predictors. The above ratio PI's are unit-less: any bias in gene copy measurements will tend to cancel out.

TABLE 5
PGA FISH Univariate Analysis

| Probe | location | p (Wilcoxon) |
|---|---|---|
| CYP24 | 20q13.2 | 0.001 |
| EXT1 | 8q24.11 | 0.002 |
| NR1D1 | 17q21.1 | 0.003 |
| MLN64 | 17q12 | 0.003 |
| FANCA | 16q24.3 | 0.004 |
| BIRC5 | 17q25.3 | 0.007 |
| ZNF144 | 17q12 | 0.008 |
| RAD21 | 8q24.11 | 0.012 |
| GRB7 | 17q12 | 0.016 |
| HEPSIN | 19q13.12 | 0.02 |
| ZNF207 | 17q11.2 | 0.03 |
| STK3 | 8q22.2 | 0.051 |
| IMPA1 | 8q21.13 | 0.062 |
| AL080059 | 8q22.1 | 0.073 |
| SMARCE1 | 17q21.2 | 0.075 |
| ANXA11 | 10q22.3 | 0.086 |
| SMARCE1 (dup) | 17q21.2 | 0.153 |
| PDCD6IP | 3p23 | 0.526 |

If no markers were associated with recurrence, on average less than one false positive would be expected at p<0.05. 11 markers were associated with recurrence at p<0.05

### Conclusions:

1. The PGA FISH™ method described here is a valid assay for assessing gene copy number in archived tissue samples.

2. The PGA FISH™ method allows assessment of gene copy number exclusively in carcinoma cells rather than stromal or inflammatory cells.

3. 13 of the 17 genes were validated to either univariately predict recurrence or to participate in multiplex patterns that are prognostic. We found a three-gene marker (NR1D1, SMARCE1, BIRC5) in a training set that was prognostic for low risk of recurrence in women with hormone receptor negative (HR−) cancers. The marker's negative predictive value was 91% in independent test sets.

4. We found a second 3-gene marker (CYP24, PDCD6IP, BIRC5) in a training set that is prognostic for low risk of recurrence in women with hormone receptor positive (HR+) cancers. The marker's negative predictive value was also 91% in independent test sets.

5. In women with hormone receptor negative, lymph node negative (HR−, N0) cancers, the first marker marker (SMARCE1, NR1D1, and BIRC5) had a NPV of 100% in a test set (0 of 4 women).

6. In women with hormone receptor positive, lymph node negative (HR+, N0) cancers, the second marker (CYP24, PDCD6IP, and BIRC5) had a NPV of 100% in a test set (0 of 15 women). The prognostic value of these genomic markers will be studied in larger numbers of women with hormone receptor positive, node negative (HR+, N0), and hormone receptor negative, node negative (HR−, N0) cancers.

It will be apparent to those of skill in the art that in each of these cases, at least one marker that did not show statistical significance as a univariate marker, pdcd6ip (43r), is present in each of the three multivariate probe sets described above. In contrast, none of the multivariate sets include both of the markers with the greatest statistical significance as univariate probes (77r (ext1) and 82 g (birc5)). While not being bound by any specific theory, we hypothesize that the combinations of markers disclosed herein as the greatest predictors of recurrence provide markers for different cellular pathways, and thus their combination provides added information over and above other combinations of markers with more significance as univariate markers, but which provide redundant information on the same or similar cellular pathways. This representation of different cellular pathways may make the combination markers presented in these examples likely candidates for predicting the effectiveness of treatment with combinations of drugs targeted as those distinct cellular pathways.

### Example 3

5 BAC DNAs were selected, those for NR1D1, SMARCE1, BIRC5, CYP24A, and PDCD6IP. The BACs were selected from the “32K human genome BAC Rearray”, maintained at the CHORI (http://bacpac.chori.org/). The sizes of the BACS in this example range from 154-178 kb. Details of individual BACs are summarized in Table 6.

TABLE 6

| Clone | Size of BAC (kb) | Chromosome Location |
|---|---|---|
| NR1D1 | 162 | 17q21.1 |
| SMARCE1 | 174 | 17q21.2 |
| BIRC5 | 178 | 17q25.3 |
| CYP24 | 171 | 20q13.2 |
| PDCD6IP | 154 | 3p23 |

The repetitive elements of the BACs were identified using the “Mask Repeat” function at the UCSC Genome Bioinformatics database, and repeat-free sequences of the BACs were obtained using the “Get DNA” function (http://genome.ucsc.edu/index.html?org=Human).

The sequence of the related BAC clone for each marker was down loaded from Human UCSC Genome browser with the repetitive sequences marked. Primers were selected from both ends of a unique sequence area of no less than 300 by length. Primers were designed for each BAC using the “Fast PCR” program.

TABLE 7
PCR primers and products

| | Number of PCR | Average size of | Number of bp in |
|---|---|---|---|
| Clone | primer pairs | PCR products | unique sequence probe |
| NR1D1 | 88 | 925 bp | 81 kb |
| SMARCE1 | 94 | 1720 bp | 81 kb |
| BIRC5 | 96 | 814 bp | 78 kb |
| CYP24 | 72 | 877 bp | 63 kb |
| PDCD6IP | 96 | 1240 bp | 60 kb |

PCR was carried out as follows:

PCR Master Mix from Promega (Catalog #M7505) was used for PCR.

Reaction buffer: 25 units/ml of Taq DNA Polymerase, in Promega's proprietary reaction buffer −(pH 8.5), 200 uM dATP, 200 uM dGTP, 200 uM dCTP, 200 uM dTTP, 1.5 mM MgCl₂.

Cycling conditions:

1. 5 minutes at 95° C.;
2. 15 seconds at 95° C.;
3. 30 seconds at 56° C.; (This temperature varies depending on the annealing temperature of the primer.)
4. 2 minutes at 72° C.;
5. go to step 2 for 30 times;
6. 3 minutes at 72° C.;

Primer concentration was 1 uM.

Template concentration:

The BAC clone concentration for the first run PCR was between 0.3 and 0.5 ng/ul.

For the second run PCR, 1/100 volume of the first run PCR reaction mix was used as template and concentration was not determined.

For a 50 ul first run PCR reaction, mix:

0.3 ul BAC template (56 ng/ul);
5 ul forward primer (10 uM);
5 ul reverse primer (10 uM);
25 ul 2×PCR master mix;
14.7 ul sterile dH₂O

And run through the above cycle and store at −20° C.

For a 50 ul second run PCR reaction, mix

0.5 ul first run PCR product;
5 ul forward primer (10 uM);
5 ul reverse primer (10 uM);
25 ul 2×PCR master mix;
14.5 ul sterile dH₂O

And run through the above cycle and store at −20° C.

The melting temperature for all the primers used ranged from 57° to 60° C. Analytical agarose gels demonstrating amplified PCR products showed that 98-99% of the reactions were successful.

The numbers of primers pairs for each BAC, the average length of the PCR products, and the total coverage of the original BAC included in the unique sequence probe are summarized in Table 8:

TABLE 8

| | complete | unique seq | amplified | % unique/ | % complete | % unique seq |
|---|---|---|---|---|---|---|
| Clones | seq bp | bp | seq bp | complete seq | seq amplified | amplified |
| PDCD6IP | 150437 | 77036 | 59000 | 0.512081469 | 39.21907509 | 76.58756945 |
| SMARCE1 | 180862 | 105440 | 79000 | 0.582985923 | 43.6797116 | 74.92412747 |
| BIRC5 | 177623 | 97050 | 76000 | 0.546381944 | 42.78725165 | 78.31014941 |
| NR1D1 | 161994 | 95161 | 80000 | 0.587435337 | 49.38454511 | 84.06805309 |
| CYP24 | 180862 | 105440 | 62000 | 0.582985923 | 34.28027999 | 58.80121396 |

The sequences of the PCR products included in the unique sequence probe are provided as follows:

| | | |
|---|---|---|
| a) | SMARCE1 USP: | SEQ ID NOS: 66-159 |
| b) | PDCD6IP USP: | SEQ ID NOS: 160-255 |
| c) | CYP24 USP: | SEQ ID NOS: 256-327 |
| d) | NR1D1 USP: | SEQ ID NOS: 328-415; and |
| e) | BIRC5 USP: | SEQ ID NOS: 416-511. |

The PCR products were purified from the un-extended primers and pooled, and then aliquots of each pool were labeled with at least one of four fluorochromes: Spectrum Orange, Spectrum Green, Spectrum Red (Vysis) or DEAC (diethylaminocoumarin-5-dUTP, PerkinElmer) using Invitrogen's BioPrime DNA Labeling Kit, according to manufacturer's instructions. Unincorporated fluorochromes were purified using YM-30 microcon columns (Millipore).

Probes were hybridized either alone, in pairs, or in triplets (ie: for 1, 2, or 3 nucleic acid targets; and thus 1, 2, or 3 different USPs) to metaphase chromosomes or to breast cancer thin sections, or to sectioned tissue culture cell lines sectioned from paraffin blocks. The hybridization buffer was 20% formamide, 10% dextransulfate, 0.9% NaCl. The probe concentrations were 40 ng/μl if labeled with Spectrum Orange, Texas Red, or DEAC Aqua, and 60 ng/μl for the Spectrum Green. The specimens were hybridized at 38° C. for 14-20 hours. After hybridization, the slides were washed with 0.1% NaCl at 60° C. for 5 minutes and then in fresh 0.1% NaCl at 60° C. for 3 minutes.

For each example, the original BAC and its derivative USP were labeled with one of the four fluorochromes co-hybridized on metaphase chromosomes. In each pair of dual labeled probes. Competitor DNA was included in these hybridizations in order to suppress the repetitive sequences contained in the parental BACs. In each of the pairs, the probes co-hybridize with equal intensity to the expected chromosome region, without hybridization to any other chromosome region, and without any specific or non specific background or artifactual hybridization to any other chromosome regions. These dual hybridizations demonstrate that there is no loss of specificity or signal quality between the labeled parental BAC and its derivative USP.

On metaphase chromosomes, the signal intensities for all probes were equal for all five probes, regardless of the fluorochrome. In all cases bright, strong signals were clearly visible on the correct chromosome, without any artifactual signal on other chromosomes. There was no background hybridization randomly distributed throughout the genome, nor were there any areas of diffuse non-specific hybridization.

Multiplex hybridization of probe triplets was also tested on 5 uM formalin-fixed, paraffin embedded, interphase breast adenocarcinoma cell line thin sections. In these hybridizations, clear distinct hybridization signals were seen, in the three unique colors expected for the fluorochromes used, without artifactual signals or interfering background. Thus there is no loss of signal quality or intensity whether the probes were hybridized singly or in triplets.

Probe quality and signal strength were not dependent on particular pairs of probes and fluorochromes. Pair-wise combinations of fluorochromes and probes that have been tested are summarized in Table 9.

Table 9 summarizes the combinations of probes with fluorochromes that have been examined. Signals quality is rated +++++ if strong clear unambiguous without background or artifactual signals.

| | Spectrum | | Spectrum | |
|---|---|---|---|---|
| Clone | Orange | Texas Red | Green | DEAC Aqua |
| NR1D1 | ++++ | ++++ | | |
| SMARCE1 | | ++++ | ++++ | |
| BIRC5 | ++++ | | ++++ | ++++ |
| CYP24 | ++++ | ++++ | ++++ | ++++ |
| PDCD6IP | ++++ | ++++ | ++++ | |

We claim:

1. A composition comprising a breast cancer biomarker, wherein the breast cancer biomarker consists of between 2 and 35 different probe sets, wherein at least 40% of the different probe sets comprise one or more isolated polynucleotides that selectively hybridize to a genomic region selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the different probe sets in total selectively hybridize to at least two of the recited genomic regions.

2. The composition of claim 1 wherein the different probe set in total selectively hybridize to at least three of the recited genomic regions.

3. A composition comprising a breast cancer biomarker consisting of between 2 and 42 different probe sets, wherein at least 40% of the different probe sets comprise one or more isolated polynucleotides that selectively hybridize to a nucleic acid according to formula 1, or its complement:
X1-X2-X3;
wherein X2 is selected from the group consisting of SEQ ID NOS: 18-65 or their complement, wherein X1 and X3 are independently 0-500 kB of human genomic nucleic acid flanking X2 in the human genome; and
wherein the different polynucleotide probe sets in total selectively hybridize to at least two non-overlapping nucleic acids according to formula 1.

4. The composition of claim 3 wherein the different polynucleotide probe sets in total selectively hybridize to at least three non-overlapping nucleic acids according to formula 1.

5. A composition comprising a breast cancer biomarker consisting of between 2 and 42 different probe sets, wherein at least 40% of the different probe sets comprise or consist of one or more isolated polynucleotides that selectively hybridize to a nucleic acid according to one of SEQ ID NO:1-17 or complements thereof wherein the different probe sets in total selectively hybridize to at least two of the recited nucleic acids according to SEQ ID NO:1-17 or complements thereof.

6. The composition of claim 5 wherein the different polynucleotide probe sets in total selectively hybridize to at least three of the recited nucleic acids according to SEQ ID NO:1-17 or complements thereof.

7. A method for classifying a breast tumor, comprising
(a) contacting a nucleic acid sample obtained from a subject having a breast tumor with a composition comprising a breast cancer biomarker according to the present invention that, in total, selectively hybridize to two or more genomic regions selected from the group consisting of 3p23, 8q21.13, 8q22.1, 8q22.2, 8q24.11, 10q22.3, 16q24.3, 17q11.2, 17q12, 17q21.1, 17q22.2, 17q25.3, 19q13.12, and 20q13.2; wherein the contacting occurs under conditions to promote selective hybridization of the polynucleotides of the probe set to the two or more genomic regions;
(b) detecting formation of hybridization complexes;
(c) determining from the detected hybridization complexes whether one or more of the genomic regions are present in an altered copy number in the nucleic acid sample; and
(d) correlating an altered copy number of two or more of the genomic regions with an increased risk for breast cancer recurrence.

8. A method for classifying a breast tumor, comprising
(a) contacting a nucleic acid sample obtained from a subject having a breast tumor with the composition according to claim 3; wherein the contacting occurs under conditions to promote selective hybridization of the polynucleotides of the probe set to a target in the nucleic acid sample;
(b) detecting formation of hybridization complexes;
(c) determining from the detected hybridization complexes whether two or more markers selected from the group consisting of SEQ ID NOS: 18-65, or complements thereof, are present at an altered copy number in the nucleic acid sample; and
(d) correlating an altered copy number of two or more markers selected from the group consisting of SEQ ID NOS: 18-65, or complements thereof with an increased risk for breast cancer recurrence.

9. The method of claim 8 wherein the determining comprises determining from the detected hybridization complexes whether three or more markers selected from the group consisting of SEQ ID NOS: 18-65, or complements thereof, are present at an altered copy number in the nucleic acid sample, and the correlating comprises correlating an altered copy number of three or more markers selected from the group consisting of SEQ ID NOS: 18-65, or complements thereof with an increased risk for breast cancer recurrence.
